(19) United States
(12) Patent Application Publication
(10) Pub. No.: US 2024/0390009 A1
(43) Pub. Date: Nov. 28, 2024

(54) INTRAVASCULAR REPERFUSION THERAPY WITH AN EXPANDABLE STRUCTURE AND ASSOCIATED DEVICES, SYSTEMS, AND METHODS

(71) Applicant: KONINKLIJKE PHILIPS N.V., EINDHOVEN (NL)

(72) Inventors: James David CEZO, COLORADO SPRINGS, CO (US); Arjen VAN DER HORST, TILBURG (NL)

(21) Appl. No.: 18/693,339

(22) PCT Filed: Sep. 20, 2022
(86) PCT No.: PCT/EP2022/076012

§ 371 Date: Mar. 19, 2024

Related U.S. Application Data
(60) Provisional application No. 63/246,919, filed on Sep. 22, 2021.

(51) Int. Cl. A61B 17/12; A61B 17/00; A61B 90/00

(52) U.S. Cl. CPC A61B 17/1204; A61B 17/00234; A61B 17/12122; A61B 17/12131; A61B 90/06; A61B 2017/00022; A61B 2017/00106; A61B 2017/00199; A61B 2017/00305; A61B 2017/00862; A61B 2017/00955; A61B 2017/1205; A61B 17/12109; A61B 2090/064

(57) ABSTRACT

A system includes an intravascular reperfusion therapy device positionable within a coronary vein to deliver reperfusion therapy to a myocardium of a heart. The intravascular reperfusion therapy device includes a catheter and a sensor. The catheter includes a plurality of arms configured to expand into the coronary vein to deliver the reperfusion therapy by obstructing blood flow in a first direction to generate back pressure in an opposite, second direction. The system includes a processor circuit that receives, from the sensor, physiological data associated with blood flow, determines, based on the physiological data, a progression of the reperfusion therapy, and controls, based on the progression of the reperfusion therapy, expansion of the plurality of arms while the intravascular reperfusion therapy device is positioned within the coronary vein such that the back pressure within the coronary vein is controlled.

### TECHNICAL FIELD

The present disclosure relates generally to reperfusion therapy, and, in particular, to monitoring and controlling the progression of the reperfusion therapy based on intravascular sensing. More specifically, physiological data representative of blood flow through a vessel may be used to determine a progression of a reperfusion therapy targeting an area of a heart of a patient and control the configuration of an expandable structure delivering the reperfusion therapy based on the determined progression.

### BACKGROUND

A percutaneous coronary intervention (PCI) may be utilized to treat a blockage (e.g., an occlusion, a lesion, a stenosis, and/or the like) within a blood vessel. The PCI may include a therapeutic procedure, such as administration of a drug, angioplasty, placement of a stent, and/or the like, that reduces a size of the blockage or opens (e.g., widens) the lumen of the affected blood vessel. To that end, PCI can restore blood flow through a blood vessel and to tissue that receives blood/oxygen via the blood vessel. Moreover, before a PCI therapy is delivered, the reduction in blood flow caused by a blockage within a blood vessel may cause tissue that receives blood/oxygen from the vessel to experience ischemia. Accordingly, PCI may restore or increase blood flow to tissue that experienced ischemia, which may restore the health of the tissue. However, even after a PCI therapy is delivered, blood/oxygen may not always suitably re-perfuse through tissue that has experienced ischemia. In particular, the increase and/or reintroduction of blood flow through the ischemic tissue may trigger an inflammatory response and/or oxidative damage, known as reperfusion injury, along with or in place of restoration of normal function of the tissue.

### SUMMARY

Disclosed herein is a system configured to evaluate (e.g., assess) and control (e.g., modify) a progress of a reperfusion therapy targeting an area of a patient's body, such as a portion of the myocardium of the heart of the patient. The system may include an intravascular reperfusion therapy device and a processing system, which may include a processor circuit. The intravascular reperfusion therapy device may be positioned within a blood vessel, such as a coronary vein, of the patient and may deliver the reperfusion therapy to the area, such as myocardium, associated with (e.g., receiving blood flow from or providing blood flow to) the blood vessel. The intravascular reperfusion therapy device may deliver the reperfusion therapy via a plurality of arms (e.g., an expandable structure), which may expand into the blood vessel to obstruct the blood vessel and thereby alter blood flow through the area. In particular, the plurality of arms, when expanded, may generate a back pressure within a coronary vein to deliver reperfusion therapy to a myocardium. Further, the intravascular reperfusion therapy device may include a sensor (e.g., a sensing component), which may sense physiological data (e.g., flow data, pressure data, and/or the like) representative of blood flow through the blood vessel. In that regard, the physiological data may indicate an effect of the reperfusion therapy on the area. To that end, the processing system may receive the physiological data from the sensor and may determine a progression of the reperfusion therapy delivered to the area. Moreover, based on the progression of the reperfusion therapy, the processing system may control (e.g., modify or maintain) the reperfusion therapy delivered by the intravascular device. In particular, the processing system may control expansion of the plurality of arms such that obstruction of the blood vessel, the generated back pressure, and delivery of the reperfusion therapy is controlled. In this way, the processing system may use a feedback loop with the sensor to adaptively monitor and control delivery of the reperfusion therapy via the expansion of the plurality of arms. In cases where the intravascular reperfusion therapy device is positioned within a coronary vein, for example, when a determined progression of the reperfusion therapy indicates that blood flow to the targeted area is improving and/or that the reperfusion therapy has achieved a particular outcome, the processing system may control the plurality of arms to gradually transition from a first degree of expansion to a second degree of expansion to decrease the venous obstruction or complete (e.g., terminate) the administration of the reperfusion therapy.

In an exemplary aspect, a system is provided. The system includes an intravascular reperfusion therapy device configured to be positioned within a coronary vein of a patient to deliver reperfusion therapy to a myocardium of a heart of the patient associated with the coronary vein, wherein the intravascular reperfusion therapy device comprises a catheter and a sensor, wherein the catheter comprises a plurality of arms configured to expand into the coronary vein to deliver the reperfusion therapy by obstructing blood flow in a first direction in the coronary vein to generate back pressure in an opposite, second direction within the coronary vein; and a processor circuit in communication with the intravascular reperfusion therapy device and configured to: receive, from the sensor, physiological data associated with blood flow through the coronary vein; determine, based on the physiological data, a progression of the reperfusion therapy delivered to the myocardium of the heart; and control, based on the progression of the reperfusion therapy, expansion of the plurality of arms while the intravascular reperfusion therapy device is positioned within the coronary vein such that the back pressure within the coronary vein is controlled.

In some aspects, to determine the progression of the reperfusion therapy, the processor circuit is configured to: determine a derivative of the physiological data with respect to time. In some aspects, to control the expansion of the plurality of arms, the processor circuit is configured to control a degree of expansion of the plurality of arms, and the degree of expansion corresponds to at least one of a quantity of expanded arms of the plurality of arms or a diameter to which the expanded arms are expanded. In some aspects, to control the expansion of the plurality of arms, the processor circuit is configured to control an oscillation of the degree of expansion between a first degree of expansion and a different, second degree of expansion. In some aspects, the processor circuit is configured to control a frequency of the oscillation. In some aspects, the processor circuit is configured to control a duty cycle of the oscillation. In some aspects, when expanded, the plurality of arms is configured to be spaced from one another such that a gap is positioned between a first and second arm of the plurality of arms. In some aspects, the intravascular reperfusion therapy device comprises a material coupled between the plurality of arms, and the expansion of the plurality of arms results in expansion of the material into the coronary vein. In some aspects, the intravascular reperfusion therapy device comprises: a first expansion state in which the plurality of arms is unexpanded; and a second expansion state in which at least one of the plurality of arms is expanded into the coronary vein. In some aspects, the plurality of arms comprise an electroactive polymer configured to deform responsive to an electrical signal, and the processor circuit is configured to control the electrical signal to transition the intravascular reperfusion therapy device from the first expansion state to the second expansion state. In some aspects, the plurality of arms comprise material configured to deform responsive to application of thermal energy, and the processor circuit is configured to control the thermal energy to transition the intravascular reperfusion therapy device from the first expansion state to the second expansion state. In some aspects, in the first expansion state, the plurality of arms is positioned within a lumen of the catheter, and, in the second expansion state, the plurality of arms is positioned distal of a distal end of the catheter. In some aspects, the plurality of arms is integrally formed in the catheter such that the intravascular reperfusion therapy device is configured to buckle a portion of the catheter from a first position to a different, second position to transition from the first expansion state to the second expansion state. In some aspects, the processor circuit is further configured to: output, to display in communication with the processor circuit, a visual representation of the progression of the reperfusion therapy. In some aspects, the sensor comprises a flow sensor, and the physiological data comprises a blood flow rate. In some aspects, the sensor comprises a pressure sensor, and the physiological data comprises pressure data. In some aspects, the sensor comprises an ultrasound transducer positioned at a distal portion of the catheter. In some aspects, the intravascular reperfusion therapy device further comprises a guidewire, and the sensor is positioned at a distal portion of the guidewire.

In an exemplary aspect, a system is provided. The system includes an intravascular reperfusion therapy device configured to be positioned within a coronary vein of a patient to deliver reperfusion therapy to a myocardium of a heart of the patient associated with the coronary vein, wherein the intravascular reperfusion therapy device comprises: a flow sensor positioned at a distal portion of the intravascular reperfusion therapy device; and a catheter comprising a plurality of arms, wherein the plurality of arms is configured to expand into the coronary vein to deliver the reperfusion therapy by obstructing blood flow in a first direction in the coronary vein to generate back pressure in an opposite, second direction within the coronary vein; and a processor circuit in communication with the intravascular reperfusion therapy device and configured to: receive, from the flow sensor, flow data associated with blood flow through the coronary vein; determine, based on the flow data, a progression of the reperfusion therapy delivered to the myocardium of the heart; and control, based on the progression of the reperfusion therapy, a degree of expansion of the plurality of arms while the intravascular reperfusion therapy device is positioned within the coronary vein such that the back pressure within the coronary vein is controlled, wherein the degree of expansion corresponds to at least one of a quantity of expanded arms of the plurality of arms or a diameter to which the expanded arms are expanded.

In an exemplary aspect, an apparatus is provided. The apparatus includes an intravascular reperfusion therapy device configured to be positioned within a coronary vein of a patient to deliver reperfusion therapy to a myocardium of a heart of the patient associated with the coronary vein, wherein the intravascular reperfusion therapy device comprises: a catheter; a plurality of arms coupled to a distal portion of the catheter, wherein the plurality of arms is configured to expand into the coronary vein to deliver the reperfusion therapy by obstructing blood flow in a first direction in the coronary vein to generate back pressure in an opposite, second direction within the coronary vein; and a control mechanism coupled to a proximal portion the catheter, wherein the control mechanism is configured to control a degree of expansion of the plurality of arms while the intravascular reperfusion therapy device is positioned within the coronary vein such that the back pressure within the coronary vein is controlled, wherein the degree of expansion corresponds to at least one of a quantity of expanded arms of the plurality of arms or a diameter to which the expanded arms are expanded. Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic view of a system, in accordance with at least one embodiment of the present disclosure.

FIG. 2 is a schematic diagram of a processor circuit, in accordance with at least one embodiment of the present disclosure.

FIG. 3A is diagram of a human heart with an obstruction, in accordance with at least one embodiment of the present disclosure.

FIG. 3B is diagram of the human heart following a percutaneous coronary intervention (PCI), in accordance with at least one embodiment of the present disclosure.

FIG. 3C is diagram of the human heart following a reperfusion therapy, in accordance with at least one embodiment of the present disclosure.

FIG. 4 is a schematic diagram of a portion of a heart, in accordance with at least one embodiment of the present disclosure.

FIG. 5 is a schematic diagram of a portion of a heart, in accordance with at least one embodiment of the present disclosure.

FIG. 6A is a diagrammatic side view of an intravascular reperfusion therapy device in a first expansion state, in accordance with at least one embodiment of the present disclosure.

FIG. 6B is a diagrammatic side view of the intravascular reperfusion therapy device in a second expansion state, in accordance with at least one embodiment of the present disclosure.

FIG. 6C is a cross-sectional view of the cut plane A-A shown in FIG. 6B, in accordance with at least one embodiment of the present disclosure.

FIG. 7 is a flow diagram of a method for evaluating a progression of a reperfusion therapy, in accordance with at least one embodiment of the present disclosure.

FIG. 8 is a schematic diagram of a portion of a heart, in accordance with at least one embodiment of the present disclosure.

FIG. 9 is a diagrammatic, schematic view of a screen display, in accordance with at least one embodiment of the present disclosure.

FIG. 10 is a schematic diagram of a portion of a heart, in accordance with at least one embodiment of the present disclosure.

FIG. 11A is a diagrammatic side view of an intravascular reperfusion therapy device in a first expansion state, in accordance with at least one embodiment of the present disclosure.

FIG. 11B is a diagrammatic side view of the intravascular reperfusion therapy device in a second expansion state, in accordance with at least one embodiment of the present disclosure.

FIG. 11C is a front view of the intravascular reperfusion therapy device in the second expansion state, in accordance with at least one embodiment of the present disclosure.

FIG. 12A is a front view of an intravascular reperfusion therapy device, in accordance with at least one embodiment of the present disclosure.

FIG. 12B is a front view of an intravascular reperfusion therapy device, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

Aspects of the present disclosure can include features described in App. No. 63/246,946, filed Sep. 22, 2021 (Atty Dkt No. 2021PF00227/44755.2210PV01), App. No. 63/246,904, filed Sep. 22, 2021 (Atty Dkt No. 2021PF00224/44755.2211PV01), App. No. 63/246,963, filed Sep. 22, 2021 (Atty Dkt No. 2021PF00228/44755.2212PV01), and App. No. 63/246,929, filed Sep. 22, 2021 (Atty Dkt No. 2021PF00226/44755.2214PV01), the entireties of which are incorporated by reference herein.

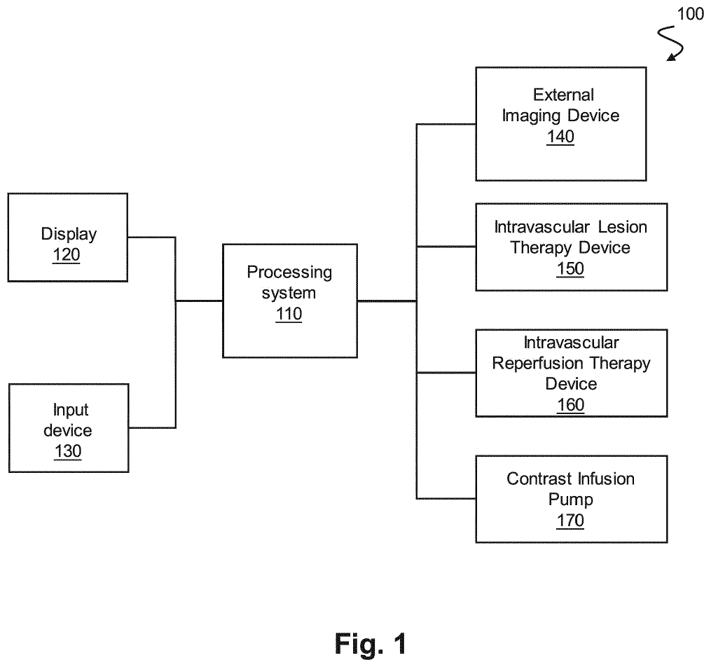

Referring to FIG. 1, shown therein is a system 100 according to an embodiment of the present disclosure. The system 100 can be configured to evaluate (e.g., assess), display, and/or control (e.g., modify) a progress of a reperfusion therapy targeting an area of a patient's body, such as a portion of the myocardium. For instance, the system 100 may be utilized to monitor and/or control reperfusion therapy such that injury to the myocardium following a percutaneous coronary intervention (PCI) is avoided or minimized, as described in greater detail below. In this regard, the system 100 may be used to assess coronary vessels and/or heart tissue (e.g., the myocardium) oxygenated by the coronary vessels. As illustrated, the system 100 includes a processing system 110 in communication with a display 120 (e.g., an electronic display), an input device 130 (e.g., a user input device), an external imaging device 140, an intravascular lesion therapy device 150 (e.g., intraluminal therapy device), an intravascular reperfusion therapy device 160 (e.g., intraluminal reperfusion therapy device), and a contrast infusion pump 170.

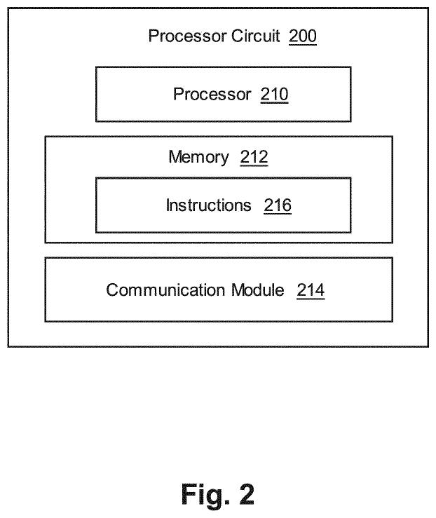

The processing system 110 is generally representative of any device suitable for performing the processing and analysis techniques disclosed herein. In some embodiments, the processing system 110 includes processor circuit, such as the processor circuit 200 of FIG. 2. In some embodiments, the processing system 110 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processor circuit (e.g., computing device) using corresponding instructions stored on or in a non-transitory computer readable medium accessible by the computing device. In some instances, the processing system 110 is a console device. Further, it is understood that in some instances the processing system 110 comprises one or a plurality of computing devices, such as computers, with one or a plurality of processor circuits. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

FIG. 2 is a schematic diagram of a processor circuit 200, according to embodiments of the present disclosure. The processor circuit 200 may be implemented in the processing system 110 of FIG. 1. As shown, the processor circuit 200 may include a processor 210, a memory 212, and a communication module 214. These elements may be in direct or indirect communication with each other, for example via one or more buses.

The processor 210 may include a central processing unit (CPU), a digital signal processor (DSP), an ASIC, a controller, an FPGA, another hardware device, a firmware device, or any combination thereof configured to perform the operations described herein. The processor 210 may also be implemented as a combination of computing devices, e.g., a combination of a DSP and a microprocessor, a plurality of microprocessors, one or more microprocessors in conjunction with a DSP core, or any other such configuration.

The memory 212 may include a cache memory (e.g., a cache memory of the processor 210), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 212 includes a non-transitory computer-readable medium. The memory 212 may store instructions 216. The instructions 216 may include instructions that, when executed by the processor 210, cause the processor 210 to perform the operations described herein with reference to the processing system 110 (FIG. 1). Instructions 216 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 214 can include any electronic circuitry and/or logic circuitry to facilitate direct or indirect communication of data between various components of the processor circuit 200 and/or the processing system 110 (FIG. 1). Additionally or alternatively, the communication module 214 can facilitate communication of data between the processor circuit 200, the display 120 (e.g., a monitor), the input device 130, the external imaging device 140, the intravascular lesion therapy device 150, the intravascular reperfusion therapy device 160, the contrast infusion pump 170, and/or the like. In that regard, the communication module 214 can be an input/output (I/O) device interface, which may facilitate communicative coupling between the processor circuit 200 and (I/O) devices, such as the input device 130. Moreover, the communication module 214 may facilitate wireless and/or wired communication between various elements of the processor circuit 200 and/or the devices and systems of the system 100 using any suitable communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G.

Turning back now to FIG. 1, the external imaging device 140 can include an x-ray system, angiography system, fluoroscopy system, ultrasound system, computed tomography (CT) system, a magnetic resonance imaging (MRI) system, other suitable imaging devices, and/or combinations thereof. The external imaging device 140 may additionally or alternatively include a nuclear medicine imaging device, such as a gamma camera or a single-photon emission computed tomography (SPECT) system, other suitable devices, and/or combinations thereof. The external imaging device 140 can be configured to acquire imaging data of anatomy, such as the heart and blood vessels, while the external imaging device 140 is positioned outside of the body of the patient. The imaging data can be visualized in the form of two-dimensional and/or three-dimensional images of the heart, blood vessel, and/or other anatomy. In some embodiments, the imaging device 140 need not be an external device that is positioned outside the patient body. For example, the imaging device 140 can be an intracardiac echocardiography (ICE) catheter that obtains images while positioned within a heart chamber. In some embodiments, the imaging device 140 may be an external device in that is it is positioned outside of the particular anatomy that is being imaged (e.g., blood vessels and/or heart), but is positioned inside the patient body. For example, the imaging device 140 can be a transesophageal echocardiography (TEE) probe that obtains images while positioned within an esophagus.

Moreover, the external imaging device 140 may obtain images of the heart that are indicative of the health of the cardiac muscle or myocardium. In particular, the external imaging device 140 can be configured to acquire imaging data that illustrates myocardial perfusion (e.g., myocardial perfusion imaging (MPI) data). For example, MPI data can be collected by imaging a radiopharmaceutical agent, such as thallium, in the patient's heart muscle using a SPECT system. Additionally or alternatively, the imaging data may be obtained by imaging a contrast agent, which may be administered to the patient's vasculature manually or via the contrast infusion pump 170, for example. In any case, the imaging data can illustrate vasculature and/or muscle mass with blood flow and/or vasculature and/or muscle mass that lack of blood flow in areas of the heart.

The contrast infusion pump 170 may administer a contrast agent, which may alter an appearance (e.g., a brightness, an intensity, a contrast) of a feature within an external imaging data, such as the external imaging data obtained by the external imaging device 140. In that regard, the contrast infusion pump 170 may be configured to administer, to the patient, a contrast agent that is radiopaque and enhances the visibility of internal fluids or structures within a patient's anatomy. In some embodiments, for example, the contrast agent absorbs external x-rays from an x-ray source, resulting in decreased exposure on an x-ray detector in conjunction with the x-ray source. The contrast agent may be of any suitable material, chemical, or compound and, before administration to the patient, may be a liquid, powder, paste, tablet, or of any other suitable form. For example, the contrast agent may include iodine-based compounds, barium sulfate compounds, gadolinium-based compounds, microbubbles, or any other suitable compounds, which may be included in a solution or suspension, for example, for administration to the patient. In some embodiments, the contrast agent may include carbon dioxide, which may be a gas. In such cases, the contrast agent may decrease absorption of the external x-rays from the x-ray source, when administered. The contrast agent may additionally be referred to as a radiocontrast agent, a contrast dye, a radiocontrast dye, a contrast material, a radiocontrast material, a contrast media, or a radiocontrast media, among other terms. Further, in some embodiments, the contrast infusion pump 170 may be configured to combine or switch between different contrast agents, which may reduce stress on the patient's body. For instance, the contrast infusion pump 170 may administer a first contrast agent for a period of time and may subsequently administer a different, second contrast agent to the patient during an imaging procedure.

The intravascular lesion therapy device 150 may be any form of device, instrument, or probe sized and shaped to be positioned within a vessel. For example, the intravascular lesion therapy device 150 is generally representative of a guide wire, a catheter, or a guide catheter. However, in other embodiments, the intravascular lesion therapy device 150 may take other forms. In that regard, the intravascular lesion therapy device 150 may be a device configured to deliver a PCI therapy to a vessel. In particular, the intravascular lesion therapy device 150 may be an intravascular guidewire or catheter configured to ablate a lesion (e.g., a blockage) within the vessel, deploy a balloon, a stent, and/or drug to a target site within the vessel, and/or the like. That is, for example, the intravascular lesion therapy device 150 may be a stent or balloon delivery device (e.g., an angioplasty device), a thrombectomy device, an atherectomy device, and/or the like. In that regard, the intravascular lesion therapy device 150 may include a coil retriever, an aspiration (e.g., suction) device, and/or the like to assist in the removal of a clot or occlusion from the patient's vessel. In some embodiments, the intravascular lesion therapy device 150 may include a laser, a blade (e.g., knife), a sanding crown, and/or any suitable device that may assist in the cutting, shaving, sanding, vaporizing, and/or removal of atherosclerotic plaque from the patient's vessel. Additionally or alternatively, the intravascular lesion therapy device 150 may be the therapy itself delivered to the vessel. More specifically, the intravascular lesion therapy device 150 may represent a stent or balloon deployed to the vessel, a drug administered intra or extravascularly (e.g., orally), and/or the like. To that end, while the intravascular lesion therapy device 150 is illustrated as being communicatively coupled to the processing system 110, embodiments are not limited thereto.

In some embodiments, the intravascular reperfusion therapy device 160 may be a device, instrument, or probe sized and shaped to be positioned within a vessel. In particular, the intravascular reperfusion therapy device 160 may be a device or instrument configured to control reperfusion of blood flow into a target tissue area (e.g., capillary bed), such as a portion of the myocardium of a patient. In some embodiments, the target tissue area may be an ischemic area and/or an area of tissue that receives reduced blood flow due to a blockage in an associated vessel (e.g., an upstream artery). As described in greater detail below, treatment (e.g., therapy) directed to the vessel associated with the blockage, such as treatment via the intravascular lesion therapy device 150, may reintroduce or increase blood flow to the target tissue area. To reduce or prevent injury to the target tissue area resulting from this increased blood flow, the intravascular reperfusion therapy device 160 may be positioned intravascularly, such as within a coronary blood vessel, and may be configured to regulate blood flow to the target tissue. In some embodiments, the reperfusion therapy can include administration of anti-inflammatory drug(s) or nitric oxide (NO) to the patient. In some embodiments, the reperfusion therapy can include cold fluid that is provided via the arterial side.

In some embodiments, one or more of the external imaging device 140, the intravascular lesion therapy device 150, the intravascular reperfusion therapy device 160, and/or the contrast infusion pump 170, are located proximate one or more of the processing system 110, the display device 120, and/or the input device 130, such as in the same procedure room. In some embodiments, one or more of the external imaging device 140, the intravascular lesion therapy device 150, the intravascular reperfusion therapy device 160, and/or the contrast infusion pump 170 are located spaced from one or more of the processing system 110, the display device 120, and/or the input device 130, such as in different procedure rooms or facilities. For example, the external imaging device 140, the intravascular lesion therapy device 150, the intravascular reperfusion therapy device 160, and/or the contrast infusion pump 170 can be part of different systems that are communicatively coupled. In that regard, the processing system 110 can be configured to acquire the data collected from the components spaced therefrom and process the data as described herein. The external imaging device 140, the intravascular lesion therapy device 150, the intravascular reperfusion therapy device 160, and/or the contrast infusion pump 170 can be configured to transmit the collected data to the processing system 110.

The system 100 includes a display device 120 that is communicatively coupled to the processing system 110. In some embodiments, the display device 120 is a component of the processing system 110, while in other embodiments, the display device 120 is distinct from the processing system 110. In some embodiments, the display device 120 is a monitor integrated in a console device or a standalone monitor (e.g., a flat panel or flat screen monitor). The processing system 110 can be configured to generate a visual display (e.g., screen display) based on imaging data from the external imaging device 140. The processing system 110 can provide (e.g., output) the screen display to the display device 120. To that end, the display device 120 may be configured to output (e.g., display) a two-dimensional image and/or a two-dimensional representation of the heart, blood vessels, and/or other anatomy, which may be included in the screen display. In some embodiments, the display device 120 is configured to output a three-dimensional graphical representation of the heart, blood vessels, and/or other anatomy. For instance, the display device 120 may be a holographic display device configured to output a three-dimensional holographic display of anatomy. Any suitable display device is within the scope of this disclosure, including self-contained monitors, projection/screen systems, head-up display systems, etc. The display device can implement principles based on moving reflective microelectromechanical systems (MEMS), laser plasma, electro-holography, etc. In some embodiments, the display device 120 is implemented as a bedside controller having a touch-screen display as described, for example, in U.S. Provisional Application No. 62/049,265, titled “Bedside Controller for Assessment of Vessels and Associated Devices, Systems, and Methods,” and filed Sep. 11, 2014, the entirety of which is hereby incorporated by reference herein.

The system 100 includes an input device 130 that is communicatively coupled to the processing system 110. The input device 130 may be a peripheral device, such as a touch sensitive pad, a touch-screen, a joy-stick, a keyboard, mouse, trackball, a microphone, an imaging device, and/or the like. In other embodiments, the user interface device is part of the display device 120, which may be a touch-screen display, for example. Moreover, a user may provide an input to the processing system 110 via the input device 130. In particular, the input device 130 may enable a user to control, via inputs to the processing system 110, one or more of the components of the system 100, such as the external imaging device 140, the intravascular lesion therapy device 150, the intravascular reperfusion therapy device 160, the contrast infusion pump 170, or the processing system 110 itself. Additionally or alternatively, the input device 130 may facilitate interaction with a screen display provided at the display device 120. For instance, a user may select, edit, view, or interact with portions of the screen display (e.g., a GUI) provided at the display device 120 via the input device 130.

The system 100 can include various connectors, cables, interfaces, connections, etc., to communicate between the elements of the intravascular lesion therapy device 150, the intravascular reperfusion therapy device 160, the processing system 110, the external imaging device 140, the display device 120, and/or the input device 130. In some embodiments, for example, the communication module 214 (FIG. 2), which may be included in the processing system 110, may include such connectors, interfaces, and/or the like. In that regard, the processing system 110 can communicate and/or control one or more components of the system 110 via mechanical and/or electromechanical signaling and/or controls. Further, the illustrated communication pathways are exemplary in nature and should not be considered limiting in any way. In that regard, it is understood that any communication pathway between the components of system 100 may be utilized, including physical connections (including electrical, optical, and/or fluid connections), wireless connections, and/or combinations thereof. In that regard, it is understood that the one or more of the components of the system 100 can communicate via a wireless connection in some instances. In some instances, the one or more components of the system 100 and/or other systems (e.g., of a hospital or health services provider) communicate via a communication link over a network (e.g., intranet, internet, telecommunications network, and/or other network).

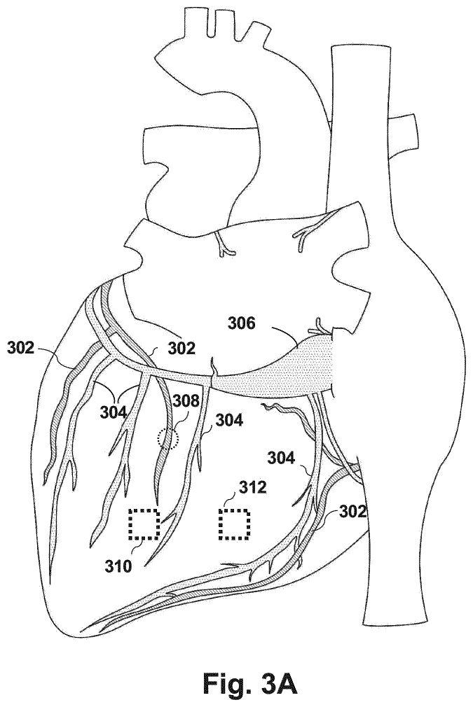

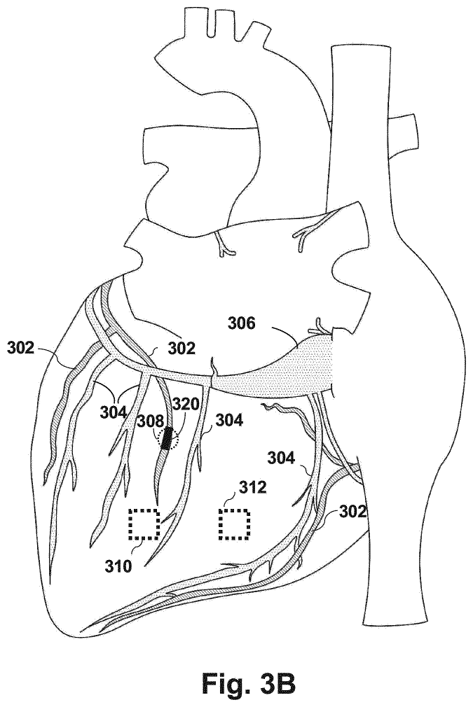

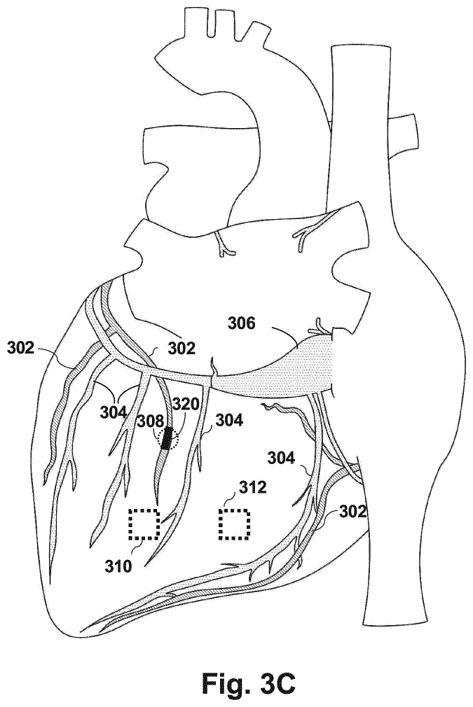

FIGS. 3A-3C illustrate a diagram of a human heart 300. As illustrated, the heart 300 includes coronary arteries 302 (illustrated with a first fill pattern) that deliver oxygenated blood to tissue, such as muscle tissue (e.g., myocardium), of the heart 300. The heart 300 further includes coronary veins 304 (illustrated with a second fill pattern), including a coronary sinus 306, that carry deoxygenated blood away from the tissue of the heart and towards a chamber (e.g., an atrium) of the heart 300.

In the diagram illustrated in FIG. 3A, a coronary artery 302 of the heart 300 includes a blockage 308 (e.g., an occlusion, a lesion, a stenosis, and/or the like). The blockage 308 may disrupt flow through the coronary artery 302. In particular, the blockage 308 may decrease the diameter of a portion of the lumen of the coronary artery 302, which may decrease the flow of blood through the portion of the lumen. As a result, a first area of tissue 310 (e.g., a portion of the myocardium) that is associated with (e.g., receives blood from) the coronary artery 302 with the blockage 308 may not receive a healthy amount of blood/oxygen. For instance, the blood/oxygen delivered to the first area of tissue 310 may not be sufficient to perfuse through (e.g., to be distributed across) the entire first area of tissue 310 in some cases. In this regard, the first area of tissue 310 may experience ischemia (e.g., a reduction in delivered blood/oxygen illustrated by a relatively dark fill pattern), which may damage the first area of tissue 310. In some embodiments, the first area 310 can be and/or include coronary artery 302 with the blockage. In some embodiments, the first area of the heart may be vasculature and/or tissue associated with (e.g., receiving blood flow from) a first vessel with a previous blockage (as opposed to a current blockage). In some embodiments, the first area may be an area that experiences or has experienced a period of reduced blood flow/ischemia. The illustrated different, second area of tissue 312 (e.g., a portion of the myocardium) may receive blood/oxygen from a different coronary artery 302 than the first area of tissue 310. In this regard, the second area of tissue 312 may remain relatively unaffected by the blockage 308. To that end, the second area of tissue 312 may receive a healthy amount of blood/oxygen, and the second area of tissue 312 may not experience ischemia. Accordingly, the second area of tissue 312 is illustrated as being healthy by a lack of the fill pattern (e.g., by a relatively brighter and/or whiter fill) shown in the first area of tissue 310. In some embodiments, the second area 312 can be and/or include coronary artery 302 without the blockage.

In some embodiments, a percutaneous coronary intervention (PCI) may be utilized to treat the blockage 308. In particular, the PCI may include a therapeutic procedure that reduces a size of the blockage 308, opens (e.g., widens) the lumen of a vessel, and/or the like to restore blood flow through the vessel (e.g., the coronary artery 302) with the blockage 308. In that regard, the PCI may include, for example, angioplasty (e.g., deploying a balloon) and positioning a stent across the stenosis to open the vessel (e.g., the coronary artery 302 with the blockage). The PCI may additionally or alternatively include thrombectomy, atherectomy, administration of a drug and/or the like. To that end, the intravascular lesion therapy device 150 (FIG. 1) may facilitate and/or provide the PCI to a vessel having a blockage (e.g., blockage 308).

FIG. 3B illustrates a diagram of the heart 300 after delivery of a therapeutic procedure (e.g., post-treatment), such as PCI. In particular, FIG. 3B illustrates a stent 320 positioned within the coronary vessel at the site of the blockage 308. However, embodiments are not limited thereto. In that regard, the PCI delivered to the coronary artery 302 or a vessel with a blockage may include any suitable combination of the therapies described above.

As described above, the stent 320 and/or another suitable PCI (e.g., therapeutic procedure) may be provided to a vessel so that an effect of a blockage on blood flow through the vessel is reduced. In this regard, the placement of the stent 320 within the coronary artery 302 (e.g., at the site of the blockage 308) may open (e.g., widen) the portion of the lumen of the coronary artery 302 with the blockage 308, which may increase blood flow through the portion lumen. Moreover, the placement of the stent 320 within the heart 300 may increase blood flow downstream of the blockage 308, such as within vasculature that receives blood flow from the portion of the lumen. In this way, the vasculature (e.g., a capillary bed) that delivers blood/oxygen to the first area of tissue 310 may receive increased blood flow, which may increase blood/oxygen delivery to the first area of the tissue 310. To that end, blood/oxygen may re-perfuse the first area of the tissue 310. Accordingly, the stent 320 may reverse or reduce the ischemia experienced by the first area of the tissue 310. In this regard, the first area of the tissue 310 is illustrated in FIG. 3B with a different fill pattern (e.g., a relatively brighter and/or lighter fill pattern) than the fill pattern illustrated in FIG. 3A to demonstrate the increased blood/oxygen supplied to the first area of the tissue 310.

In some cases, blood/oxygen may not suitably perfuse through tissue associated with an occluded vessel (e.g., a vessel with a blockage), such as the first area of tissue 310, after delivery of a PCI therapy. For example, in some cases, the introduction and/or increase of blood flow to tissue that has experienced ischemia may result in reperfusion injury (e.g., ischemia-reperfusion injury). In particular, the returned blood flow may trigger an inflammatory response and/or oxidative damage along with or in place of restoration of normal function of the tissue. Inflammation, damage resulting from inflammation, and/or the oxidative damage may obstruct the flow of blood/oxygen within the tissue (e.g., within a capillary bed associated with the tissue). Accordingly, blood/oxygen may not be distributed throughout (e.g., perfuse through) the tissue at a healthy level even after the delivery of a PCI therapy. For instance, blood may preferentially flow through a first portion of the tissue lacking inflammation and/or damage and may flow through a second portion of the tissue with inflammation and/or damage to a lesser degree. As a result, the second portion of the tissue may continue to receive blood flow below a healthy level. In this regard, the first area of the tissue 310 is illustrated in FIG. 3B with a different fill pattern (e.g., a relatively darker fill pattern) than the second area of the tissue 312 (e.g., a healthy area of tissue) to demonstrate that the stent 320 alone may not fully restore the health and/or functioning of the first area of the tissue 310.

Turning now to FIG. 3C, in some cases, the reperfusion of blood/oxygen within ischemic tissue may be further assisted and/or controlled by reperfusion therapy. To that end, FIG. 3C illustrates a diagram of the heart 300 after delivery of the PCI therapy and a reperfusion therapy, such as a therapy delivered by the intravascular reperfusion therapy device 160. In particular, FIG. 3C illustrates a diagram of the heart 300 following delivery of a reperfusion therapy targeting the first area of tissue 310. According to techniques described in greater detail below, reperfusion therapy may be delivered by the intravascular reperfusion therapy device 160 to reduce or minimize injury at and/or to improve blood flow to tissue where blood is re-perfusing (e.g., an area of tissue receiving an increase in blood flow), such as the first area of tissue 310. In particular, reperfusion therapy may affect a distribution of blood flow through the targeted tissue such that blood flow perfuses (e.g., distributes to) and/or increases throughout the tissue, including through areas of the tissue that are inflamed or have oxidative damage. Accordingly, delivery of the reperfusion therapy targeting an area of tissue may restore blood flow to a healthy amount or an amount exceeding the blood flow resulting from the PCI therapy alone. For instance, in the illustrated embodiment, the health and/or functioning of (e.g., the blood flow to) the first area of tissue 310 is shown as being fully restored by the reperfusion therapy, as indicated by the fill pattern of the first area of tissue 310 matching the second area of tissue 312. In some embodiments, however, the reperfusion therapy may restore the health and/or functioning of (e.g., the blood flow to) tissue to a level greater than a level resulting from the PCI but less than a level at an area of tissue, such as the second area of tissue 312, that was relatively unaffected by a blockage (e.g., associated with a different vessel than the vessel having the blockage). Particular mechanisms for controlling, using one or more components of the system 100, the reperfusion of an area of tissue associated with (e.g., configured to receive blood/oxygen from) a vessel that receives a PCI therapy (e.g., a vessel that has an occlusion) and/or otherwise receives an increase in blood flow are described herein.

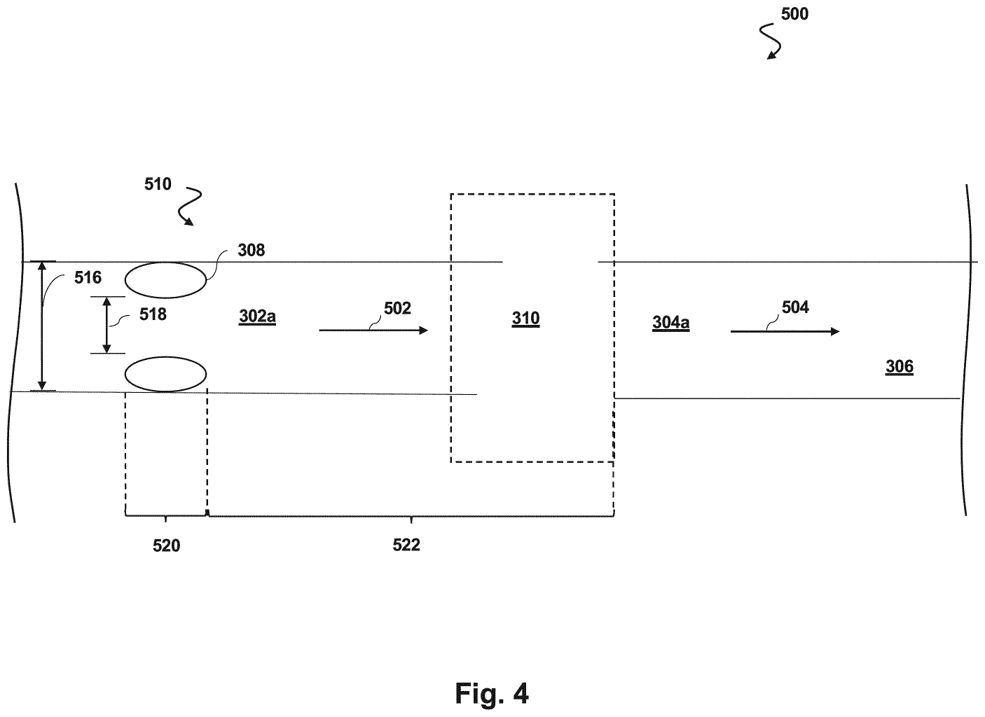

FIG. 4 illustrates a schematic diagram of a portion 500 of a heart of a patient. FIG. 4 may correspond to a detailed view of a portion of the heart 300 illustrated in FIG. 3A. More specifically, FIG. 4 illustrates a coronary artery 302, which includes a blockage 308 (e.g., a stenosis) and is arranged to provide blood/oxygen to an area of tissue 310, as indicated by the arrow 502. A coronary vein 304 is arranged to carry deoxygenated blood away from the area of tissue 310, as indicated by the arrow 504, and is illustrated as being in fluid communication with the coronary sinus 306.

As described above with respect to FIGS. 3A-3B, the blockage 308 may disrupt blood flow through the coronary artery 302. In particular, the blockage 308 may reduce a diameter of a lumen of the coronary artery 302 from a first diameter 516 to a smaller, second diameter 518 within the portion 520 of the vessel where the blockage 308 is located. This reduction in diameter may increase the resistance and/or the impedance to blood flowing through the portion 520, which may reduce blood/oxygen delivered distal of the blockage (e.g., within the distal area 522 including a portion of the coronary artery 302 and the area of tissue 310). In this regard, the area of tissue 310 (e.g., a portion of the myocardium) may experience ischemia (e.g., a reduction in delivered blood/oxygen), which may damage the area of tissue 310.

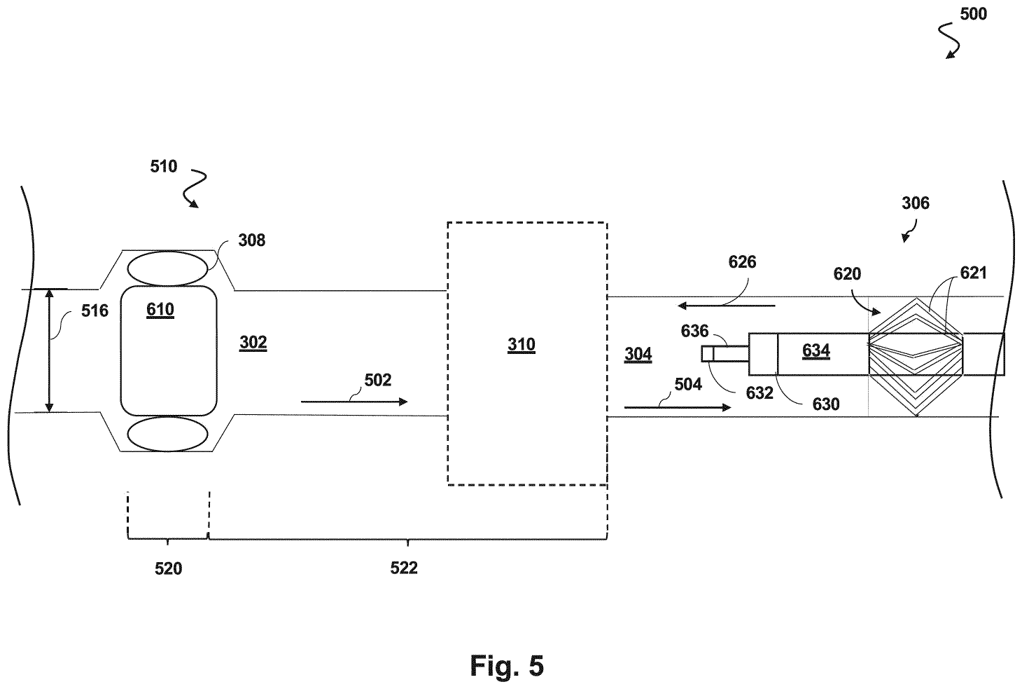

FIG. 5 is a schematic diagram of the portion 500 of the heart during and/or following delivery of a PCI therapy 610 and/or during delivery of a reperfusion therapy. FIG. 5 may correspond to a detailed view of a portion of the heart 300 illustrated in FIG. 3B. The PCI therapy 610 may be delivered by the intravascular lesion therapy device 150 and/or the PCI therapy 610 may be the intravascular lesion therapy device 150 itself. For instance, as described above with respect to FIG. 1, the PCI therapy 610 may involve ablation of the blockage 308, deployment of a balloon (e.g., angioplasty), stent, and/or drug, thrombectomy, atherectomy, and/or the like. In that regard, the PCI therapy 610 may involve a therapeutic procedure that widens the diameter available for blood flow in the portion 520 having the blockage 308 from the diameter 518 (FIG. 4). That is, for example, the PCI therapy 610 may involve widening the diameter of the lumen of the coronary artery 302, as shown. Additionally or alternatively, a size of the blockage 308 may be reduced (e.g., ablated or removed) to increase the diameter available for blood flow within the portion 520. Further, the PCI therapy 610 may involve the placement of a physical device, such as a stent, within the coronary artery 302. In some embodiments, the PCI therapy 610 may involve the use of a device (e.g., a guidewire or catheter) that is removed from the coronary artery 302 when the PCI therapy 610 is completed. In that regard, the illustrated representation of the PCI therapy 610 positioned within the coronary artery 302 is intended to be exemplary and not limiting.

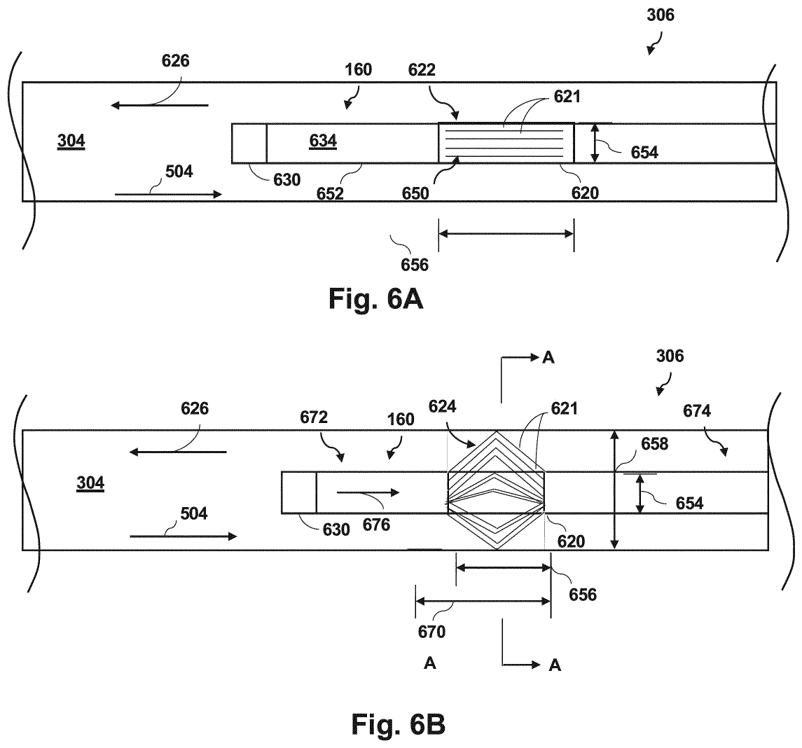

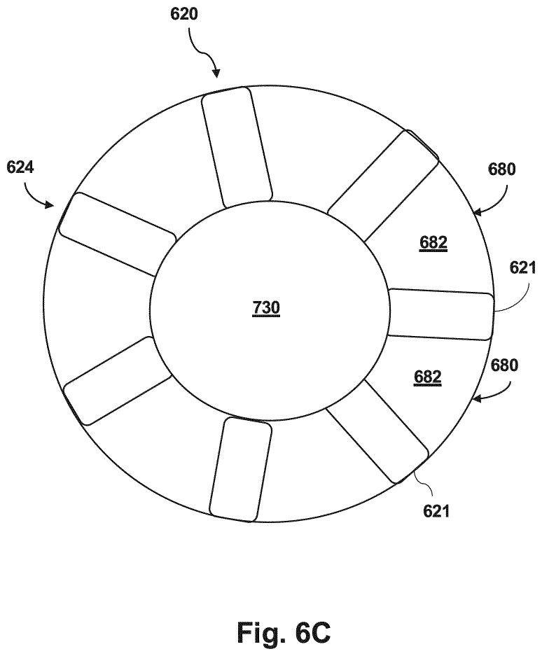

FIG. 5 further illustrates the intravascular reperfusion therapy device 160, which is shown as including an expandable basket 620 and a sensor 630. The intravascular reperfusion therapy device 160 may deliver reperfusion therapy targeting (e.g., in association with) the area of tissue 310. To that end, the intravascular reperfusion therapy device 160 is shown as being positioned at and delivering reperfusion therapy via a venous side (e.g., within the coronary sinus 306) of the area of tissue 310. In some embodiments, reperfusion therapy delivered to a venous side of the area of tissue 310 may involve obstruction of vasculature on the venous side of the first area of tissue 310. For instance, the one or more of the arms 621 of the expandable basket 620 may be configured to expand to deliver the reperfusion therapy. Such expansion of the arms 621 is illustrated and described in greater detail with respect to FIGS. 6A-C. By obstructing the vasculature with the expandable basket 620, the intravascular reperfusion therapy device 160 may increase pressure on the venous side of the tissue, which may encourage better (e.g., increased and/or more even) distribution of blood flow through the area of tissue 310, especially the damaged or inflamed portions of the area of tissue 310. In that regard, the intravascular reperfusion therapy device 160 is shown as generating a back pressure and/or back flow in a direction, indicated by arrow 626, opposite the direction, indicated by arrow 504, of blood flow through the coronary vein.

As further illustrated, the intravascular reperfusion therapy device 160 may include one or more flexible elongate members, such as a catheter 634 and/or a guidewire 636, which may extend through a portion of the catheter 634. In some embodiments, the expandable basket 620 may be positioned at a distal portion of the catheter 634. The intravascular reperfusion therapy device 160 may further include one or more sensors (e.g., sensing components). For instance, a first sensor 630 may be positioned at the distal portion of the catheter 634, such as at a distal end of the catheter 634 or spaced from and adjacent to the distal end of the catheter 634. In particular, the first sensor 630 may be positioned distal of the expandable basket 620 on the catheter 634. That is, for example, the first sensor 630 may be positioned between the area of tissue 310 and the expandable basket 620. Additionally or alternatively, the intravascular reperfusion therapy device 160 may include a second sensor 632. The second sensor 632 may be positioned on the guidewire 636, such as on a distal portion of the guidewire 636, as illustrated. The second sensor 632 may additionally or alternatively be positioned on the catheter 634. For instance, the second sensor 632 may also be positioned distal of the expandable basket 620. That is, for example, the second sensor 632 may be positioned between the area of tissue 310 and the expandable basket 620.

While both a catheter 634 and a guidewire 636 are illustrated, embodiments are not limited thereto. In some embodiments, for example, the intravascular reperfusion therapy device 160 may be implemented with only a catheter 634 or with only a guidewire 636. Similarly, the illustrated embodiment including both the first sensor 630 and the second sensor 632 is intended to be illustrative and not limiting. In that regard, the intravascular reperfusion therapy device 160 may be implemented with any suitable number of sensors, such as a single sensor, two, three, four sensors, and/or the like.

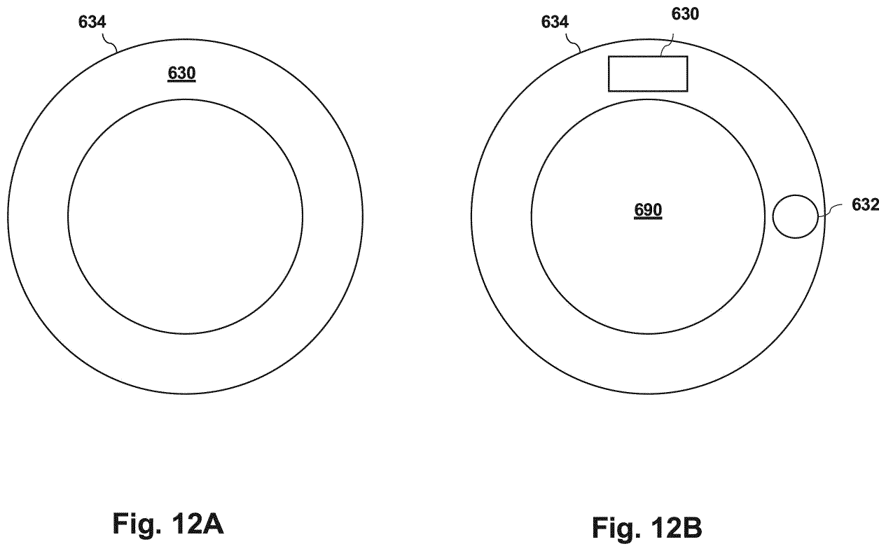

In some embodiments, the one or more sensors (e.g., 630 and 632) of the intravascular reperfusion therapy device 160 may be configured to sense (e.g., obtain) physiological data from within a blood vessel of the patient, such as a coronary vein 304 and/or the coronary sinus 306. In that regard, the physiological data may be associated with blood flow through the blood vessel. For instance, the one or more sensors may obtain flow data associated with a flow rate or flow velocity of blood through the blood vessel, pressure data associated with a blood pressure within the blood vessel, and/or the like. To that end, the one or more sensors may include a flow sensor configured to obtain the flow data, a pressure sensor configured to obtain the pressure data, and/or the like. In some embodiments, the intravascular reperfusion therapy device 160 may additionally or alternatively include an imaging device configured to obtain intravascular imaging data, such as intravascular ultrasound (IVUS) imaging data, optical coherence tomography (OCT) imaging data, and/or the like. The imaging data may be used to determine pressure data, flow data, and/or characteristics of the blood vessel, such as a diameter of the vessel, which may be useful for determining the pressure and/or flow data. Additionally or alternatively, the intravascular reperfusion therapy device 160 may include a sensor configured to determine and/or monitor a diameter or a level of expansion of the expandable basket 620 such that a level of obstruction of the blood vessel may be determined and/or monitored. In some embodiments, the one or more sensors of the intravascular reperfusion therapy device 160 may thus be the same or different from one another. As an illustrative example, the first sensor 630 may be a flow sensor, while the second sensor 632 may be a pressure sensor or vice versa. As another example, the first sensor 630 and the second sensor 632 may each be pressure sensors or may each be flow sensors. Details of the configuration of the one or more sensors are provided below with respect to FIGS. 12A-12B.

FIGS. 6A-C illustrate an exemplary intravascular reperfusion therapy device 160 that includes one or more sensors (e.g., 630 and/or 632), as well as an expandable basket 620. In particular, FIG. 6A is a diagrammatic side view of the intravascular reperfusion therapy device 160 in a first expansion state 622, FIG. 6B is a diagrammatic side view of the intravascular reperfusion therapy device 160 in a different, second expansion state 624, and FIG. 6C illustrates a cross-sectional view of the cut plane A-A shown in FIG. 6B.

As illustrated in FIG. 6A, the expandable basket 620 may be positioned on and/or formed in a flexible elongate member, such as the catheter 634, of the intravascular reperfusion therapy device 160. For instance, the arms 631 of the expandable basket 620 may be defined by slits 650 (e.g., cuts, gaps, and/or openings) formed in a body 652 of the catheter 634. That is, for example, the expandable basket 620 may be integrally formed with the catheter 634. Additionally or alternatively, the slits 650 may be formed in a sleeve coupled to the body 652 of the catheter.

In the illustrated first expansion state 622, which may correspond to the expandable basket 620 being fully collapsed (e.g., unexpanded), the expandable basket 620 may have a length 656 with respect to a longitudinal axis of the intravascular reperfusion therapy device 160. Moreover, the arms 621 of the expandable basket 620 may be positioned proximate one another and a size of the slits 650 may be minimized. In some embodiments, a diameter (e.g., height with respect to a lateral axis of the intravascular reperfusion therapy device 160) may be defined by the position (e.g., configuration) of the arms 621. In that regard, in the first expansion state 622, the arms 621 may be positioned such that the expandable basket 620 has a diameter 654, which may be substantially similar to a diameter of the catheter 634. Accordingly, obstruction of a blood vessel (e.g., a coronary vein 304) by the expandable basket 620 in the first expansion state 622 may be relatively low in comparison with the obstruction by the expandable basket 620 in other expansion states, which may correspond to relatively greater diameters of the expandable basket 620.

In some embodiments, the expandable basket 620 and/or the arms 621 may be formed from a variety of materials, including, by way of non-limiting example, plastics, polytetrafluoroethylene (PTFE), polyether block amide (PEBAX), thermoplastic, polyimide, silicone, elastomer, polymers, resins, carbon fiber, metals, such as stainless steel, titanium, superelastic or shape-memory alloys such as Nitinol, composite materials, and/or other biologically compatible materials. In particular, the arms 621 may be formed with a material and/or a configuration that is deformable. That is, for example, the arms 621 may be formed from a material configured to change shape or configurations in response to an electrical signal, thermal energy, pressure (e.g., a mechanical force), and/or the like. As an illustrative example, the arms 621 may be formed as a bimetallic strip configured to hold a first shape (e.g., configuration) for a first range of temperatures and to transition to a different, second shape (e.g., configuration) for a different, second range of temperatures (e.g., responsive to the application or dissipation of thermal energy). As another example, the arms 621 may be formed from an elastic material configured to bend from a first shape to a second shape when a pressure is applied to the arms 621 and/or the expandable basket 620 and to return to the first shape when the pressure is no longer applied to the arms 621. Additionally or alternatively, the arms may be formed from an electroactive polymer (EAP), which may exhibit a shape change in response to an electrical signal. Further, in some embodiments, the arms 621 may additionally or alternatively be formed with joints or other features suitable to alter the shape of the arms 621 responsive to an electrical signal, the application or dissipation of thermal energy, and/or a mechanical force. In any case, the arms 621 may be configured to change positions and/or configurations (e.g., shapes) from the positions and configurations shown in the first expansion state 622.

In some embodiments, altering the shape or configuration of the arms 621 (e.g., using an electrical signal, thermal energy, or a mechanical force) may transition the expandable basket 620 from the first expansion state 622 to a second expansion state, as illustrated in FIG. 6B. In particular and with reference now to FIG. 6B, to transition from the first expansion state 622 to the second expansion state 624, the arms 621 of the expandable basket 620 may deform (e.g., bend, expand, and/or the like) such that the arms 621 are spaced from one another and the expandable basket 620 expands (e.g., extends) within the blood vessel. To that end, the arms 621 are illustrated as increasing the diameter of the expandable basket 620 from the diameter 654 to the diameter 658, which may substantially match a diameter of the blood vessel (e.g., the coronary vein 304). In this way, the expanded arms 621 in the second expansion state 624 may obstruct blood flow through the blood vessel to a greater degree than in the first expansion state 622. Because the different configurations (e.g., diameters) of the expandable basket 620 correspond to different degrees of obstruction, the configuration of the expandable basket 620 corresponds to an amount of back pressure generated in the direction (indicated by arrow 626) opposite the direction (indicated by arrow 504) of blood flow. In that regard, the configuration (e.g., diameter) of the expandable basket 620 may correspond to a level (e.g., degree) of reperfusion therapy and/or the aggressiveness of the reperfusion therapy delivered to a target area of tissue (e.g., the area of tissue 310). Moreover, while the expandable basket 620 is illustrated as being arranged in the first configuration 622 and the second configuration 624, it may be appreciated that the expandable basket 620 may be configured to transition between any suitable number of configurations, which may provide a corresponding number of different degrees of obstruction of the vasculature. To that end, the obstruction of the vasculature and resulting reperfusion therapy may be coarsely or finely controlled based on the relative differences between the different configurations of the expandable basket 620.

As further illustrated, transitioning the expandable basket 620 from the first expansion state 622 to the second expansion state 624 may shorten (e.g., reduce) a length of the expandable basket 620 from the length 656 to the length 670. For instance, the expandable basket 620 may buckle from the first expansion state 622 to the second expansion state 624. In this way, a distance between two portions of the intravascular reperfusion therapy device 160, such as the portion 672 (e.g., a distal portion) and the portion 674 (e.g., a proximal portion) may be reduced when the expandable basket 620 is in the second expansion state 624 in comparison with the first expansion state 622. For example, as indicated by arrow 676, the portion 672 may be moved to a closer proximity with the portion 674 or vice versa.

In some embodiments, the intravascular reperfusion therapy device 160 may include a mechanical actuator that may control movement of the portion 672 and/or the portion 674 (e.g., the expansion state of the intravascular reperfusion therapy device). For instance, the mechanical actuator may be coupled to the portion 672 and may be configured to pull the portion 672 along the direction indicated by arrow 676 while the portion 674 is maintained in a relatively constant position to transition the intravascular reperfusion therapy device 160 to the second expansion state 624. Additionally or alternatively, the mechanical actuator may be coupled to the portion 674 and may be configured to push the portion 674 towards the portion 672 to transition the intravascular reperfusion therapy device 160 to the second expansion state 624. That is, for example, movement of the portion 672 and/or the portion 674 may compress the expandable basket 620 and cause the arms 621 to expand into the second expansion state 624. As an illustrative example, the mechanical actuator may include a knob or gears, wiring, and/or the like coupled to and configured to control the movement of the portion 672 and/or the portion 674.

Further, in some embodiments, the expansion state of the intravascular reperfusion therapy device 160 may be controlled via management of thermal energy and/or control of electrical signals supplied to the expandable basket 620. For instance, the arms 621 may be formed from a material that is configured to deform to different shapes, such as the first and second shapes illustrated in FIG. 6A and FIG. 6B, respectively, under different thermal conditions (e.g., different temperatures), and the intravascular reperfusion therapy device 160 may be configured to deliver thermal energy to the expandable basket 620 to control the shape of the arms 621. For instance, the intravascular reperfusion therapy device 160 may be configured to circulate coolant to the expandable basket 620 via one or more conduits (e.g., tubing) and/or the intravascular reperfusion therapy device 160 may include or be coupled to a heat pump or other components configured to deliver thermal energy to the expandable basket 620. As another example, the arms 621 may be formed from an electroactive polymer (EAP), which may exhibit a shape change in response to an electrical signal, and the intravascular reperfusion therapy device 160 may be include one or more electrical components and/or electronics (e.g., electrical circuitry) configured to supply the electrical signal to the expandable basket 620. In the above examples of expansion state control of the intravascular reperfusion therapy device via application of thermal energy or an electrical signal, the deformation of the arms 621 may cause movement of the portion 674 and/or the portion 672. In that regard, it may be appreciated that the expansion state of the expandable basket 620 may be controlled by directly controlling a shape of the expandable basket 620 and/or by indirectly controlling the shape of the expandable basket 620 via control of positioning of the portion 672 and/or the portion 674.

In some embodiments, the expandable basket 620 may be expanded to different degrees, which may correspond to different levels of obstruction (e.g., area of obstruction) within a blood vessel (e.g., a coronary vein 304 or a coronary artery 302). For instance, the first expansion state 622 and the second expansion state 624 may cause different levels of obstruction resulting from the differences in the diameter (e.g., 654 and 658, respectively) of the expandable basket 620 between the two states. Additionally or alternatively, the different degrees of expansion may correspond to a quantity of the arms 621 that are expanded within the blood vessel. For instance, in some embodiments, a first subset of arms 621 may be expanded to a first diameter within the blood vessel, while a second subset of arms 621 may remain unexpanded or may be expanded to a different, second diameter within the blood vessel. To that end, individual or combinations of the arms 621 may be selectively activated via mechanical, electrical, or thermal energy in some embodiments. In this way, the degree of expansion of the expandable basket 620 and the corresponding degree of obstruction to blood flow caused by the expandable basket 620 may be controlled. To that end, the back pressure generated in the direction (indicated by arrow 626) opposite the direction (indicated by arrow 504) of blood flow and the resulting reperfusion therapy may correspond to the quantity of arms 621 of the expandable basket 620 that are expanded. Moreover, while the expandable basket 620 is illustrated as being arranged in the first expansion state 622 and the second expansion state 624, it may be appreciated that the expandable basket 620 may be configured to expand according to any number of degrees of expansion (e.g., combinations of quantity of arms 621 expanded and the diameter to which the expanded arms 621 expand).

As better illustrated in FIG. 6C, expansion of the expandable basket 620 to the second expansion state 624 may cause gaps 680 to be formed and/or widened from the slits 650. That is, for example, the arms 621 may be spaced from one another by the gaps 680. Further, in some embodiments, the gaps 680 may include empty space (e.g., may be unfilled), which may allow blood to flow through the gaps 680. Accordingly, in such embodiments, even when the expandable basket 620 is expanded to a diameter matching a diameter of the blood vessel, as shown in FIG. 6B, the expandable basket 620 may only partially obstruct blood flow through a blood vessel (e.g., the coronary vein 304). In some embodiments, a material 682, such as a polymer, metal, composite material, and/or other biologically compatible material, may be coupled between the arms 621. More specifically, the material 682 may be coupled between the arms 621 such that expansion of the arms 621 (e.g., to the second expansion state 624) expands the material into the blood vessel. In particular, the material 682 may be arranged between the arms 621 such that the gaps 680 are partially or totally obstructed by the material. The material 682 may additionally or alternatively prevent blood flow through the lumen 690 of the catheter 634. Further, in cases where the material fully obstructs the gaps 680 when the expandable basket 620 is expanded to a diameter matching a diameter of the blood vessel, the expandable basket 620 may thus fully obstruct blood flow within the blood vessel.

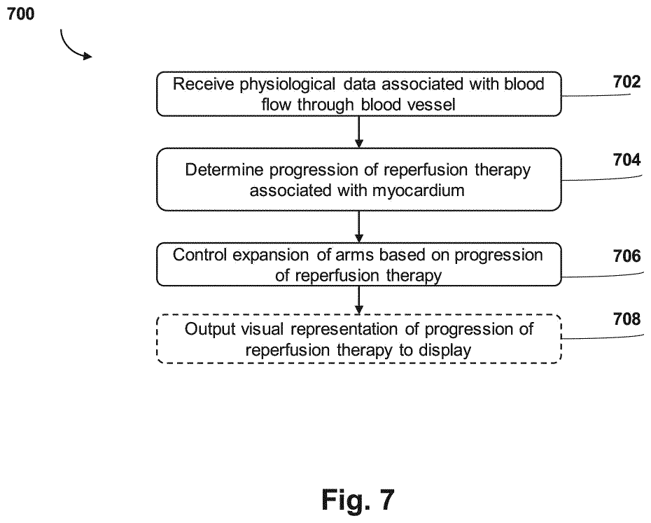

In some embodiments, the expandable basket 620 may be oscillated between different expansion states to deliver reperfusion therapy. For instance, the expandable basket 620 may be oscillated between the first expansion state 622, which may fully obstruct the vasculature, and the second expansion state 624, which may partially obstruct the vasculature. In some embodiments, the intravascular reperfusion therapy device 160 and/or the processing system 110 may be configured to oscillate the expandable basket 620 according to a set pattern (e.g., combination of frequency, duty cycle, duration, and/or the like). For instance, the processing system 110 may include instructions (e.g., instructions 216) to deliver a predetermined reperfusion therapy via a set (e.g., predetermined) control (e.g., oscillation) of the expandable basket 620. Additionally or alternatively, the processing system 110 may be configured to control the delivery of the reperfusion therapy (e.g., via control of expansion of the expandable basket 620) based on a user input, which may be received via the input device 130, for example. Moreover, in some embodiments, the processing system 110 may be configured to monitor a progression of the reperfusion therapy and adaptively control the delivery of the reperfusion therapy based on the progression of the reperfusion therapy. For instance, as described with respect to FIG. 7, the processing system 110 may control the delivery of the reperfusion therapy based on information (e.g., physiological data) received from the one or more sensors (e.g., the first sensor 630 and/or the second sensor 632) of the intravascular reperfusion therapy device 160. That is, for example, the processing system 110 may control delivery of the therapy via a feedback loop with the intravascular reperfusion therapy device 160.

In some embodiments, the intravascular perfusion therapy device 160 can be a physician-controlled apparatus. In some embodiments, the intravascular perfusion therapy device 160 does not include an integrated sensor or control by the processing system 110. The intravascular reperfusion therapy device 160 can include the catheter 634. The expandable basket 620 with the multiple arms 621 can be coupled to the catheter 634, e.g., at a distal portion of the flexible elongate member of the catheter 634. The intravascular reperfusion therapy device 160 can include a control mechanism (e.g., switch, knob, etc.) coupled to the catheter 634, e.g., at a proximal portion of the flexible elongate member of the catheter 634. For example, the control mechanism can be part of a handle at the proximal end of the intravascular reperfusion therapy device 160 and/or the catheter 634. The control mechanism can control a degree of expansion of the plurality of arms 621 while the intravascular reperfusion therapy device is positioned within the coronary vein 304 such that the back pressure within the coronary vein 304 is controlled. The degree of expansion corresponds to a quantity of expanded arms 621 and/or a diameter to which the expanded arms 621.

In some embodiments, the intravascular reperfusion therapy device 160 can include one or more control lines that are mechanical components, such as a pullwire or tendon. For example, one or multiple of such lines can be mechanically coupled to the actuator of the intravascular reperfusion therapy device 160 that controls movement of the portion 672 and/or the portion 674, and thus the expansion state of the intravascular reperfusion therapy device. The line(s) and the actuator thus mechanically open and/or close the valve 621. A user can actuate the control mechanism to pull (increase tension) and/or release (decrease tension) the line(s), which causes the valve 621 to open and/or close (e.g., via the actuator). In this manner, the line(s) physically changes to respond to user control to open and/or close the valve 621. The line(s) can be made of, e.g., metal or polymer. For example, the line(s) can be a braided metal or polymer wire that is made up of multiple strands.

While the present disclosure describes embodiments of the intravascular reperfusion therapy device including an expandable basket or expandable multi-arm structure (with or without material in between the arms), it is understood that the intravascular reperfusion therapy device can include any suitable structure that selectively restricts blood flow and/or generates back pressure. For example, the structure can be the expandable basket or expandable multi-arm structure. In some embodiments, the structure can be a controllable pump and a lumen for fluid (e.g., saline, oxygen). The structure can be an obstruction in some embodiments. For example, the obstruction can be an expandable structure (e.g., open/close valve or valve stent, expandable balloon) that can be controlled to be in one state (closed valve or expanded balloon restricting blood flow) and another state (open valve or contracted balloon allowing blood flow).

FIG. 7 is a flow diagram of a method 700 of evaluating (e.g., assessing) and controlling (e.g., modifying) a progress of reperfusion of a tissue, according to aspects of the present disclosure. In some embodiments, the method 700 may be used to control a reperfusion therapy, which may be delivered via a device, such as the intravascular reperfusion therapy device 160 of FIG. 1. In particular, the method 700 may be used to control an intravascular reperfusion therapy device positioned within a blood vessel associated with an area of tissue that reperfusion therapy is targeting, such as the device 160 illustrated in FIG. 5. As an illustrative example, the blood vessel may be a coronary blood vessel, such as a coronary vein, and the area of tissue may be a myocardium (e.g., muscular tissue of the heart). The method 700 may additionally or alternatively be used to control a reperfusion therapy targeting microvascular disease (e.g., affecting capillary beds, for example) in any portion of a patient's anatomy (e.g., within or separate from the heart) and/or nonobstructive coronary artery disease. Further, in some cases, the area of tissue may be an area of tissue that receives an increase in blood flow, which may result from administration of a therapy, such as a PCI therapy.

As illustrated, the method 700 includes a number of enumerated steps, but embodiments of the method 700 may include additional steps before, after, or in between the enumerated steps. In some embodiments, one or more of the enumerated steps may be omitted, performed in a different order, or performed concurrently. The steps of the method 700 can be carried out by any suitable component within the system 100 and all steps need not be carried out by the same component. In some embodiments, one or more steps of the method 700 can be performed by, or at the direction of, a processor circuit of the system 100, including, the processing system 110 (e.g., the processor 210 (FIG. 2)) or any other component. For the purposes of example, method 700 is described with respect to features illustrated in FIGS. 1, 2, 4, 5, 6A-C, and 8. The description with respect to these features is intended to be illustrative and not limiting.

At step 702, the method 700 involves receiving physiological data associated with blood flow through a blood vessel. In particular, the physiological data may be associated with blood flow through a blood vessel that is associated with an area of tissue targeted for reperfusion therapy. As an illustrative example, the blood vessel may be a coronary blood vessel, such as a coronary vein (e.g., the coronary sinus 306 or another suitable vein), and the area of tissue may be a myocardium (e.g., muscular tissue of the heart) associated with the coronary vein. For instance, deoxygenated blood may flow from the myocardium to the associated coronary vein, as illustrated by the relationship of the area of tissue 310 with the coronary vein 304 and the coronary sinus 306 shown in FIGS. 4-5. For the purposes of example, the method 700 is described herein with respect to a myocardium (e.g., the area of tissue 310) and a coronary vein, such as the coronary sinus 306. However, embodiments are not limited thereto.

The processing system 110 may receive the physiological data from the one or more sensors (e.g., the first sensor 630 and/or the second sensor 632) of the intravascular reperfusion therapy device. Moreover, the one or more sensors may obtain the physiological data from within the blood vessel (e.g., intravascularly), as illustrated by the arrangement of the intravascular reperfusion therapy device 160 shown in FIG. 5. To that end, the processing system 110 may receive the physiological data while the one or more sensors are positioned within the blood vessel.

In some embodiments, the physiological data may include flow data, such as a flow rate or flow velocity of blood through the blood vessel. Additionally or alternatively, the physiological data may include pressure data, such as measurements of blood pressure within the blood vessel. Further, the physiological data may include data associated with the anatomy of the blood vessel, such as a diameter of the blood vessel, a distance between a component of the intravascular reperfusion therapy device and a wall of the blood vessel, and/or the like. In some embodiments, the physiological data may additionally or alternatively include intravascular imaging data, such as IVUS imaging data, OCT imaging data, and/or the like, obtained by the intravascular reperfusion therapy device 160.

In some embodiments, the processing system 110 may further receive external imaging data associated with the blood vessel and/or the area of tissue associated with the blood vessel. For instance, the processing system 110 may receive imaging data obtained by the external imaging device 140, such as one or more include x-ray images, CT images, MRI images, SPECT images, external ultrasound images, and/or the like. The external imaging data may include contrast (e.g., may be obtained using contrast), which may be delivered by the contrast infusion pump 170, for example, such that blood flow and/or anatomical features may be visibly identified. In that regard, physiological data associated with the blood flow through the blood vessel may further be received and/or determined via external imaging data.

At step 704, the method 700 may involve determining a progression of a reperfusion therapy. That is, for example, a progression of a reperfusion therapy targeting an area of tissue (e.g., a portion of the myocardium) associated with the blood vessel corresponding to the physiological data may be determined. In that regard, the reperfusion therapy may be a therapy delivered by the intravascular reperfusion therapy device 160 to an area of tissue 310 (e.g., a portion of the myocardium). Moreover, the reperfusion therapy may regulate blood flow to the targeted tissue in some manner. In particular and as illustrated in FIG. 5, the intravascular reperfusion therapy device 160 may generate a back pressure and/or back flow in a direction (e.g., indicated by arrow 626) opposite the flow of blood (e.g., indicated by arrow 504) within the blood vessel. The back pressure and/or back flow may change a pressure and/or resistance/impedance at the area of tissue 310, which may affect the distribution of blood flowing into (e.g., indicated by the arrow 502) the microvasculature (e.g., capillary bed) of the area of tissue 310. In that regard, the reperfusion therapy may be delivered such that blood suitably perfuses throughout the area of tissue 310, including to portions of the tissue 310 that are inflamed or damaged.

In some embodiments, determining a progression of the reperfusion therapy (e.g., at step 704) may involve determining a characteristic of blood flow through the blood vessel (e.g., a coronary vein 304) and/or through the area of tissue 310 (e.g., a portion of the myocardium). In that regard, the processing system 110 may determine the progression of the reperfusion therapy delivered via the intravascular reperfusion therapy device 160 based on physiological data, such as physiological data received from the intravascular reperfusion therapy device 160 and/or external imaging data received from the external imaging device 140. For instance, based on the physiological data (e.g., pressure data, flow data, external imaging data, and/or the like), the processing system 110 may determine a flow velocity, a flow rate, and/or a pressure of blood within the blood vessel. The processing system 110 may additionally or alternatively determine a volume of blood and/or a resistance/impedance to blood flow within the blood vessel using the physiological data. For instance, the processing system 110 may determine the resistance and/or the impedance based on flow data and pressure data, in some embodiments.

The processing system 110 may relate a determined value (e.g., level) and/or change thereof of flow velocity, flow rate, pressure, volume, resistance/impedance, and/or the like to a progression of the reperfusion therapy. In general, the processing system 110 may associate a progression of a reperfusion therapy that produces values of such parameters or changes in these values indicating greater and/or increasing blood flow through the blood vessel as relatively effective (e.g., as having a positive and/or intended effect). Further, the processing system 110 may associate a progression of a reperfusion therapy that produces values of such parameters or changes in these values indicating lower, unchanging, and/or decreasing blood flow through the blood vessel as relatively ineffective (e.g., as having a negative and/or unintended effect).

More specifically, in some embodiments, the processing system 110 may compare the determined values of such parameters (e.g., parameters associated with blood flow) to one or more thresholds or other values. As an illustrative example, a first range of values may correspond to healthy blood flow conditions, while a second range of values may correspond to unhealthy blood flow conditions. To that end, responsive to determining that a value of a parameter falls within the first range (e.g., satisfies a threshold), the processing system 110 may determine that the reperfusion therapy is complete, and responsive to determining that a value of a parameter falls within the second range (e.g., fails to satisfy a threshold), the processing system 110 may determine that additional reperfusion therapy may be delivered to the target area of tissue. In some embodiments, the first range of values and/or the second range of values may be determined on historical data associated with other patients, historical data associated with the patient, data associated with a healthy blood vessel (e.g., a vessel relatively unaffected by or not associated with the blockage 308) of the patient, and/or the like.

Additionally or alternatively, the processing system 110 may determine the change in value of such parameters over the delivery of the reperfusion therapy (e.g., over time) and may relate this change to the progression of the reperfusion therapy. In some embodiments, for example, the processing system 110 may compare a current value of a parameter with a set of one or more previous values of the parameter and/or determine a derivative of the parameter with respect to time. For instance, the processing system 110 may determine a derivative of the physiological data and/or of a parameter determined based on the physiological data with respect to time. To that end, the processing system 110 may determine whether a value of the physiological data and/or the parameter is increasing, decreasing, or relatively unchanging. Moreover, the processing system 110 may determine a rate of change of the value of the physiological data and/or the parameter. In this way, the processing system 110 may determine whether the reperfusion therapy is improving blood flow to the area of tissue 310 and/or the rate at which the reperfusion therapy is impacting the blood flow to the area of tissue 310. That is, for example, the processing system 110 may determine an efficacy and/or an efficiency of the reperfusion therapy.

In some embodiments, the processing system 110 may compare the change in the value of the parameter and/or the derivative of the parameter to one or more thresholds to determine a progression of the reperfusion therapy. For instance, a first range of values may correspond to the reperfusion therapy demonstrating improvement for (e.g., a positive effect on) the area of tissue, a second range may correspond to the reperfusion therapy demonstrating worsening blood flow conditions at the area of tissue, and a third range may correspond to the reperfusion therapy demonstrating having no effect or a relatively small effect on the area of tissue. Such thresholds may additionally or alternatively be used to quantify a degree of an effect the reperfusion therapy has on the tissue area. Moreover, the processing system 110 may use the physiological data and/or derivatives thereof to determine when the reperfusion therapy is complete by determining whether the value of one or more parameters has reached a healthy threshold or has plateaued at a maximum value.

Further, the progression of the reperfusion therapy may additionally or alternatively be determined based on external imaging data obtained by the external imaging device 140. For instance, in some embodiments, the processing system 110 may determine changes in blood flow (e.g., changes in flow velocity, flow rate, and/or the like) through a vessel over time (e.g., before, during, and/or after delivery of reperfusion therapy) based on external imaging data (e.g., from the external imaging device 140). For instance, using imaging processing techniques, the processing system 110 may identify a change in a blood flow through a blood vessel based on a change in an intensity, contrast, brightness, color, and/or the like of an area (e.g., an image element and/or a pixel) of external image data obtained with contrast. An example of image processing includes conducting a pixel level analysis to evaluate whether there is a change in the color of the pixel between different images in external image data captured over different time points. Continuing with this example, for external imaging data obtained with a contrast that decreases exposure of blood (e.g., on an x-ray detector in conjunction with the x-ray source), a change in pixel color from a lighter color (e.g., gray or white) to a darker color (e.g., black) within different external images may indicate an increase in blood flow (e.g., blood perfusion) through an area (e.g., blood vessel and/or tissue) associated with the pixel, which may correspond to a progression of the reperfusion therapy providing improvement to a patient's health. A change in pixel color from a darker color to a lighter color, on the other hand, may indicate a decrease in blood flow through the area, which may correspond to a progression of the reperfusion therapy providing a negative effect on a patient's health. Moreover, as similarly described with the values of physiological data and/or parameters associated with the data, changes in blood flow and/or external imaging data characteristics (e.g., intensity, contrast, brightness, and/or the like) identified based on external imaging data may be compared to one or more thresholds and/or may be further evaluated to determine the progression of the reperfusion therapy and/or a rate of the progression of the reperfusion therapy.

At step 706, the method 700 may involve controlling the reperfusion therapy based on the determined progression of the reperfusion therapy (e.g., based on the physiological data associated with the blood flow through the blood vessel). In some embodiments, for example, the processing system 110 may control and/or instruct the intravascular reperfusion therapy device 160 based on the progression of the reperfusion therapy. For instance, in the case of reperfusion therapy being delivered venously, the intravascular reperfusion therapy device 160 may be configured to obstruct a coronary vein (e.g., the coronary sinus 306) via expansion of the expandable basket 620. In particular, the processing system 110 may oscillate the expandable basket 620 between different degrees (e.g., amounts and/or levels) of expansion, such as the first expansion state 622 and the second expansion state 624 illustrated in FIGS. 6A-6C.

In that regard, the expandable basket 620 may be oscillated between fully and partially occluding a blood vessel or between occluding the blood vessel to a first extent and occluding the blood vessel to a different, second extent (e.g., between two different degrees of expansion). To control or adjust the reperfusion therapy, the processing system 110 may modify a degree of expansion of the expandable basket 620 (e.g., a diameter and/or a quantity of expanded arms 621 of the basket 620) and/or a frequency, duty cycle, duration, and/or the like of the oscillation of the expandable basket 620. More specifically, adjusting the expansion (e.g., the degree and/or oscillation of expansion) of the expandable basket 620 may alter the level (e.g., amount) of back pressure and/or back flow generated by the intravascular reperfusion therapy device 160 in the direction (indicated by arrow 626) opposite the direction (indicated by arrow 504) of blood flow in the coronary blood vessel. As an illustrative example, increasing the quantity of arms 621 expanded and/or the diameter to which the expandable basket 620 is expanded may increase the obstruction of blood flow caused by the expandable basket 620, increasing the back pressure and/or back flow generated by the intravascular reperfusion therapy device 160.

In some embodiments, based on the determined progression of the reperfusion therapy indicating that the progression is relatively poor, not effective, and/or that blood flow is not improving in the blood vessel, the processing system 110 may control the expandable basket 620 to increase back pressure resulting on the venous side of the tissue (e.g., increase venous obstruction). In that regard, the processing system 110 may instruct the expandable basket 620 such that a blood flow (e.g., blood perfusion) through the area of tissue 310 is increased. As an illustrative example, increasing a period of the duty cycle that the expandable basket 620 is expanded to the second expansion state 624 with respect to the first expansion state 622 may increase the back pressure and/or back flow, as well as an aggressiveness of the reperfusion therapy. The processing system 110 may additionally or alternatively control the expansion of the expandable basket 620 according to a first configuration (e.g., degree of expansion and/or oscillation characteristics) responsive to determining that the reperfusion therapy is relatively poor, ineffective, and/or that blood flow through the vessel is not improving. Further, based on the determined progression of the reperfusion therapy indicating that the progression is relatively good, effective, and/or that the blood flow is improving in the blood vessel, the processing system 110 may instruct the expandable basket 620 to maintain a current therapy (e.g., oscillation pattern and/or degree of expansion) and/or to gradually terminate (e.g., ease off) delivery of the therapy. Continuing with the above example, decreasing the period of the duty cycle that the expandable basket 620 is expanded to the second expansion state 624 may decrease the back pressure and/or back flow, as well as an aggressiveness of the reperfusion therapy. Additionally or alternatively, the processing system 110 may control the expansion of the expandable basket 620 according to a different, second configuration (e.g., degree of expansion and/or oscillation characteristics) responsive to determining that the reperfusion therapy is relatively good, effective, and/or that blood flow through the vessel is improving. In general, the processing system 110 may adjust one or more characteristics of the intravascular reperfusion therapy device 160 operation (e.g., configuration of the expandable basket 620) based on a feedback loop with the intravascular reperfusion therapy device 160 and the determined progression of the reperfusion therapy. In that regard, it may be appreciated that steps of the method 700 may be repeated, such that the processing system 110 may continually determine a current progression of the reperfusion therapy (e.g., based on updated physiological data) and adaptively adjust the operation of the intravascular reperfusion therapy device 160 and/or the configuration of the expandable basket 620.

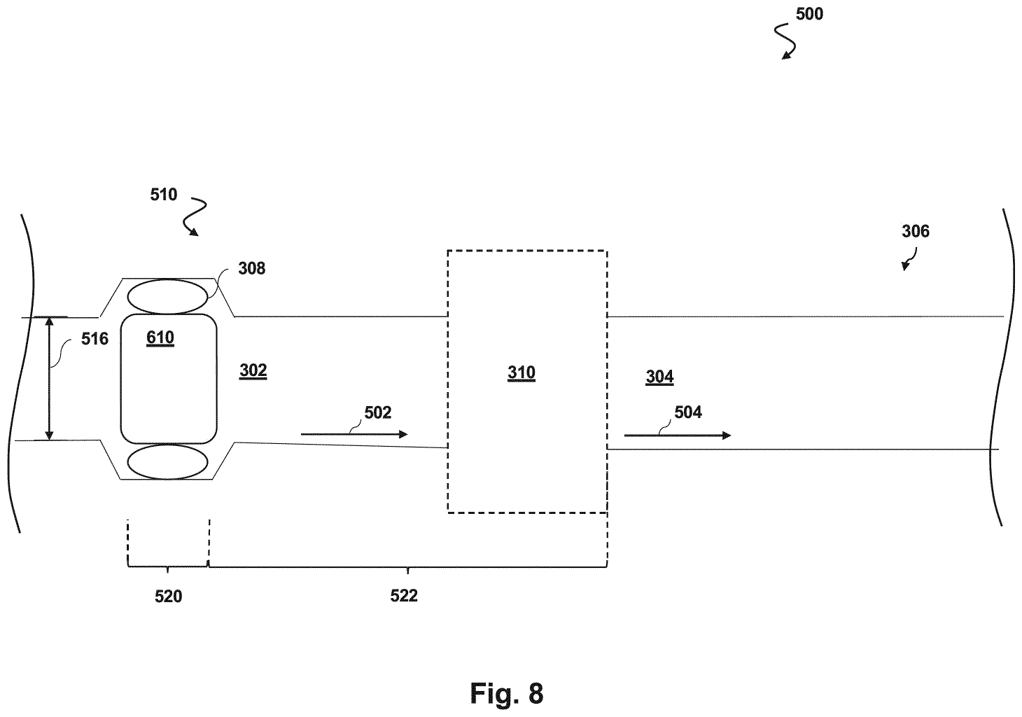

In some embodiments, responsive to determining that the blood flow through the first area has reached a healthy level (e.g., satisfies a threshold), has reached a level substantially similar to blood flow through the second area, has plateaued at a maximum value, the processing system 110 may determine that the progression of the reperfusion therapy has reached completion. In such cases, the processing system 110 may control the intravascular reperfusion therapy device 160 to terminate delivery of the reperfusion therapy. For instance, the processing system 110 transition or maintain the expandable basket 620 in the first expansion state 622 to terminate the delivery of the reperfusion therapy (e.g., responsive to determining the therapy has reached completion). FIG. 8 provides an illustrative example of the portion 500 of the heart after reperfusion therapy is complete, as illustrated by the absence of the intravascular reperfusion therapy device 160. In that regard, FIG. 8 may correspond to a detailed view of a portion of the heart 300 illustrated in FIG. 3C. As described above, when reperfusion therapy is complete, the blood flow in the first area of tissue 310 may be greater than a blood flow amount following a PCI therapy and/or may be substantially similar to the blood flow in healthy tissue, such as tissue unaffected and/or not associated with a vessel including a blockage.

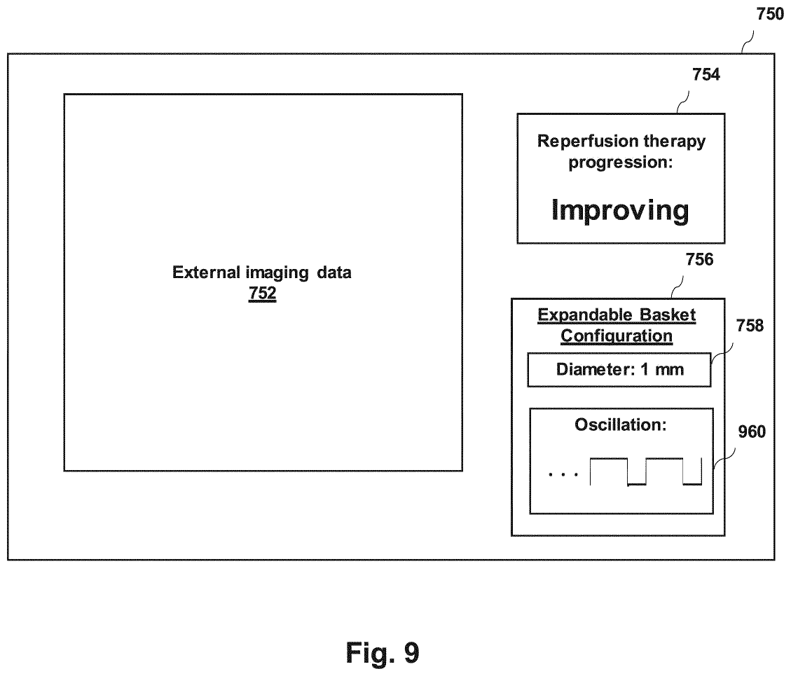

With reference again to FIG. 7, at step 708, the method 700 may optionally involve (as indicated by the dashed lines) outputting a visual representation of the progression of the reperfusion therapy to a display. For instance, the processing system 110 may output a screen display (e.g., a graphical user interface (GUI)) including a representation of the progression of the reperfusion therapy to the display device 120. An example of a screen display including a representation of the progression of the reperfusion therapy is illustrated and described with respect to FIG. 9.

FIG. 9 illustrates a screen display 750, which includes a graphical representation of external imaging data 752, a visual representation of the progress of the reperfusion therapy 754, and a visual representation of the configuration of the expandable basket 756 (e.g., a diameter and/or a degree of expansion of the expandable basket 620). The external imaging data 752 may be obtained by the external imaging device 140, as described above, and may be output to the display 120 by the processing system 110. The external imaging data 752 may depict the area of tissue 310, a coronary vein 304 associated with the area of tissue 310 (e.g., the coronary sinus 306), and/or a coronary artery 302 associated with the area of tissue 310. Moreover, the external imaging data 752 may include contrast in some embodiments. In some embodiments, the delivery and/or progress of a reperfusion therapy may be visualized via the external imaging data 752.

In some embodiments, the representation of progress of the reperfusion therapy 754 may include a visual representation of the physiological data and/or a value of a parameter determined based on the physiological data. In that regard, the visual representation may include a representation of blood flow through the blood vessel. In some embodiments, the visual representation of the progress of the reperfusion therapy 754 may include a numerical representation, a graph, chart, or plot, a textual representation, one or more symbols, and/or the like. As further illustrated, in some embodiments, the determined progression of the reperfusion therapy (e.g., at step 704) may be represented textually as a particular status, such as “improving,” “worsening,” “no change,” “complete,” and/or the like. In some embodiments, each status may correspond to a different threshold or range of values of the physiological data, parameters determined based on the data, derivations, external imaging data, and/or the like. For instance, as described above, the status “improving” may correspond to a first range of values for physiological data and/or an associated parameter that correspond to improvement for the area of tissue 310, the status “worsening” may correspond to a second range of values for the physiological data and/or associated parameter that correspond to worsening blood flow conditions associated with the area of tissue 310, the status “no change” may correspond to a third range of values for the physiological data and/or the associated parameter that correspond to the reperfusion therapy demonstrating no effect or a relatively small effect on the area of tissue 310, and the status “complete” may correspond to a fourth range of values for the physiological data and/or the associated parameter that correspond to a healthy level of blood flow associated with the area of tissue 310 and/or to a plateaued maximum value. Further, visual representation of the progress of the reperfusion therapy 754 may additionally or alternatively be indicated with respective symbols, colors, and/or the like associated with the statuses “improving,” “worsening,” “no change,” “complete,” and/or the like. Moreover, the screen display 1000 may include external imaging data 1002, such as one or more static images or an image stream. In some embodiments, navigating through the static images (e.g., using the input device 130) or watching a progression of the image stream may provide an indication of the progress of the reperfusion therapy, as the visual appearance of contrast delivered to the area of tissue will change based on an impact of the reperfusion therapy.

The visual representation of the configuration of the expandable basket 756 may display a visual representation of a diameter of the expandable basket 758 (e.g., a degree of expansion of the expandable basket 620), a visual representation of an oscillation pattern of the expandable basket 760, and/or the like. In some embodiments, the visual representation of the diameter of the expandable basket 758 may be a visual representation of a set diameter, such as a diameter the processing system 110 controls the expandable basket 620 to be expanded to, a diameter a user selects (e.g., via a user input at the input device 130) the expandable basket 758 to expand to, and/or the like. To that end, the processing system 110 may determine the visual representation of the diameter 758 based on the set diameter determined by the processing system 110 for control of the expandable basket 620 (e.g., at step 706 of the method 700) and/or a diameter selected by a user. The visual representation of the diameter 758 may additionally or alternatively be a visual representation of an actual diameter of the expandable structure 620, which may be sensed by the intravascular reperfusion therapy device 160 (e.g., via the one or more sensors 630, 632, for example) and/or determined based on the external imaging data 752. In some embodiments, for example, at least a portion of the expandable structure 620 may be radiopaque. Accordingly, the expandable structure 620 may be visualized via external imaging data 752, and the processing system 110, using the external imaging data 752 and image processing techniques (e.g., pixel level image processing), may determine the actual diameter of the expandable structure 620. Thus, the illustrated visual representation of the diameter 758 may represent the set or the actual diameter of the expandable structure 620. Moreover, while a single diameter is illustrated, embodiments are not limited thereto. In that regard, a visual representation of the actual diameter and the set diameter may be output to the display 120 in some embodiments. As further illustrated, the visual representation of the oscillation pattern of the expandable basket 760 may include a waveform of the oscillation pattern. Additionally or alternatively the visual representation of the configuration of the expandable basket 756 may include a numerical representation, a graph, chart, or plot, a textual representation, one or more symbols, and/or the like. Moreover, the visual representation of the configuration of the expandable basket 756 may include a visual representation of the quantity of arms 621 expanded. In some embodiments, the passage or obstruction of blood flow caused by the expandable basket 620 may additionally or alternatively be visualized via the external imaging data 752 (e.g., obtained with contrast).

Based on the visual representation output to the screen display, a user, such as a physician or clinician, may monitor the progression of the reperfusion therapy. Moreover, in some embodiments, the user may also control or modify the reperfusion therapy. For instance, the reperfusion therapy may be further controlled based on one or more user inputs, which may be received via the input device 130, for example. To that end, a user may provide an input at the input device 130, and the processing system 110 may control operation of the intravascular reperfusion therapy device 160 based on the input.

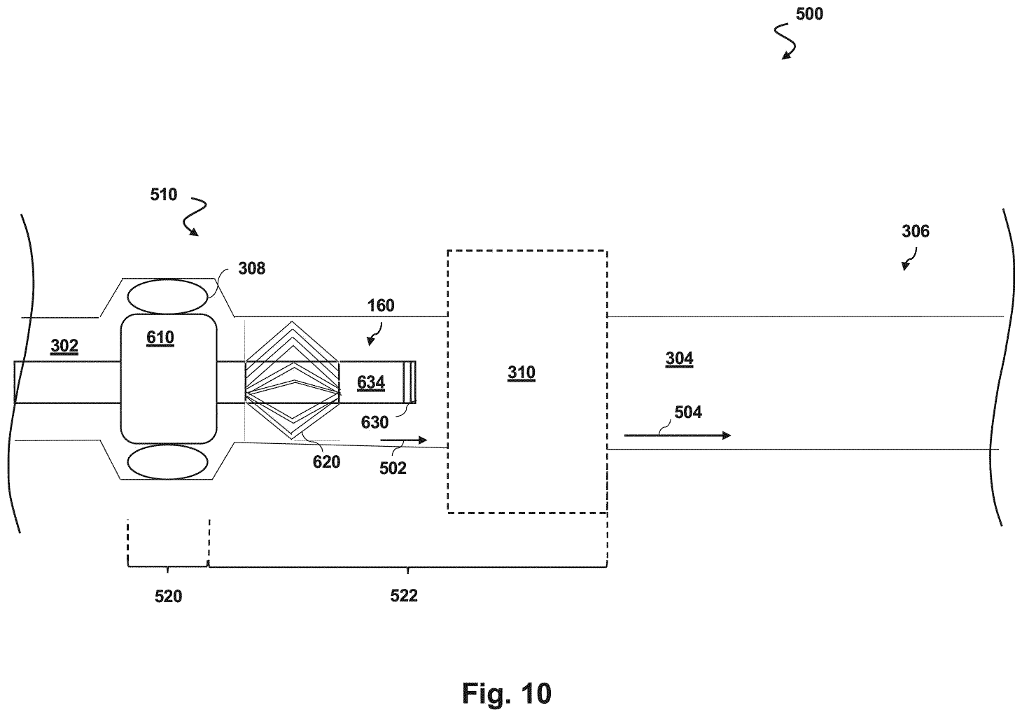

Turning to FIG. 10, a schematic diagram of the portion 500 of the heart during and/or following delivery of a PCI therapy 610 and/or during delivery of a reperfusion therapy is shown. In that regard, FIG. 10, like FIG. 5, may correspond to a detailed view of a portion of the heart 300 illustrated in FIG. 3B. However, while the reperfusion therapy is illustrated in FIG. 5 as being supplied within the venous vasculature (e.g., the coronary sinus 306), the delivery of reperfusion therapy illustrated in FIG. 10 is supplied via the arterial vasculature, such as within the coronary artery 302. In that regard, while the method 700 of FIG. 7 is described with respect to venous delivery of reperfusion therapy, the method 700 may additionally or alternatively be employed with respect to arterial delivery of reperfusion therapy, as described below.

As illustrated in FIG. 10, to deliver reperfusion therapy arterially (e.g., on the arterial side of the area of tissue 310), the intravascular reperfusion therapy device 160 may be positioned within the coronary artery 302. In particular, the intravascular reperfusion therapy device 160 may be positioned such that the one or more sensors (e.g., the first sensor 630 and/or the second sensor 632) are disposed between the expandable basket 620 and the area of tissue 310. In that regard, the processing system 110 may receive physiological data associated with blood flow through a portion of the coronary artery 302 distal of the blockage 308 (e.g., distal area 522) at step 702 of the method 700 (FIG. 7). Further, because characteristics of the blood flow through the coronary artery 302 may vary in comparison with the blood flow through the coronary veins 304, the physiological data and/or parameters associated with blood flow derived from the physiological data may be compared against different thresholds or otherwise related to a progression of a reperfusion therapy (e.g., at step 704 of the method 700) differently than the corresponding data during venous reperfusion therapy. In general, reperfusion therapy targeting the area of tissue 310 may increase blood flow through the area of tissue 310 by, for example, reducing resistance and/or impedance to flow within the area of tissue 310. Accordingly, physiological data demonstrating that blood flow through the area of tissue 310 is increasing may indicate a progression of a reperfusion therapy that is effective and/or achieving an intended effect, while physiological data demonstrating that blood flow through the area of tissue 310 is decreasing or unchanging may indicate a progression of a reperfusion therapy that is effective and/or achieving an intended effect.

The intravascular reperfusion therapy device 160 may be the same as or different from the intravascular lesion therapy device 150. In that regard, the intravascular reperfusion therapy device 160 may be configured to deliver the reperfusion therapy after a PCI therapy 610 is delivered and/or the intravascular reperfusion therapy device 160 may deliver both the PCI therapy 610 as well as reperfusion therapy. As an illustrative example, the intravascular reperfusion therapy device 160 may deploy a stent and/or perform another suitable procedure for the PCI therapy 610 and may subsequently be used to deliver reperfusion therapy. In that regard, to deliver the reperfusion therapy, the expandable basket 620 may be co-located with the blockage 308 and/or the site where the PCI therapy 610 is delivered. In some embodiments, the expandable basket 620 may be positioned between the portion 520 of the vessel where the blockage 308 and/or PCI therapy 610 (e.g., a stent) is located and the area of tissue 310. That is, for example, the expandable basket 620 may be positioned distal of the blockage 308 and/or the PCI therapy 610 (e.g., a stent) (e.g., within the distal area 522), as illustrated. Additionally or alternatively, the expandable basket 620 may be positioned proximal of the portion 520 of the vessel where the blockage 308 and/or PCI therapy 610 (e.g., a stent) is located.

In cases where the reperfusion therapy is delivered arterially, the intravascular reperfusion therapy device 160 may be configured to obstruct arterial vasculature (e.g., the coronary artery 302) at varying degrees to control the flow of blood into the area of tissue 310. As an illustrative example, the intravascular reperfusion therapy device 160 may employ the expandable basket 620 to obstruct the coronary artery 302 to a first degree (e.g., fully obstructs), as indicated by the second expansion state 624, and as the expandable basket is transitioned to the first expansion state 622 (e.g., as the diameter and/or number of expanded arms 621 of the expandable basket 620 is reduced), increased blood flow may be provided to the area of tissue 310. On the other hand, expanding the expandable basket 620 to a greater degree may decrease blood flow to the area of tissue. In this way, blood flow to the tissue 310 may be gradually increased or decreased via control of the expandable basket 620 (e.g., by the processing system 110). To that end, based on the progression of the reperfusion therapy, the processing system 110 may control the reperfusion therapy by adjusting a degree of obstruction (e.g., expansion) within the vasculature caused by the expandable basket 620 and/or by controlling a rate of expansion and/or collapse of the expandable basket 620 (e.g., at step 706 of the method 700). In some embodiments, for example, gradually increasing blood flow through the tissue 310 may minimize or prevent reperfusion injury from occurring and/or may reverse an effect of reperfusion injury. In that regard, in cases where the intravascular reperfusion therapy device 160 is used to deliver the PCI therapy 610, the expandable basket 620 may be expanded during delivery of the PCI therapy 610, and subsequently, the processing system 110 may control the expandable basket 620 to gradually reintroduce blood flow to the tissue 310 (e.g., to gradually collapse). Further, as described above, the processing system 110 may vary the degree of expansion of the expandable basket 620 and/or the rate of change between expansion states based on a feedback loop with the intravascular reperfusion therapy device 160 (e.g., based on the sensed physiological data). To that end, mechanisms to adjust reperfusion therapies described herein are intended to be illustrative and not limiting.

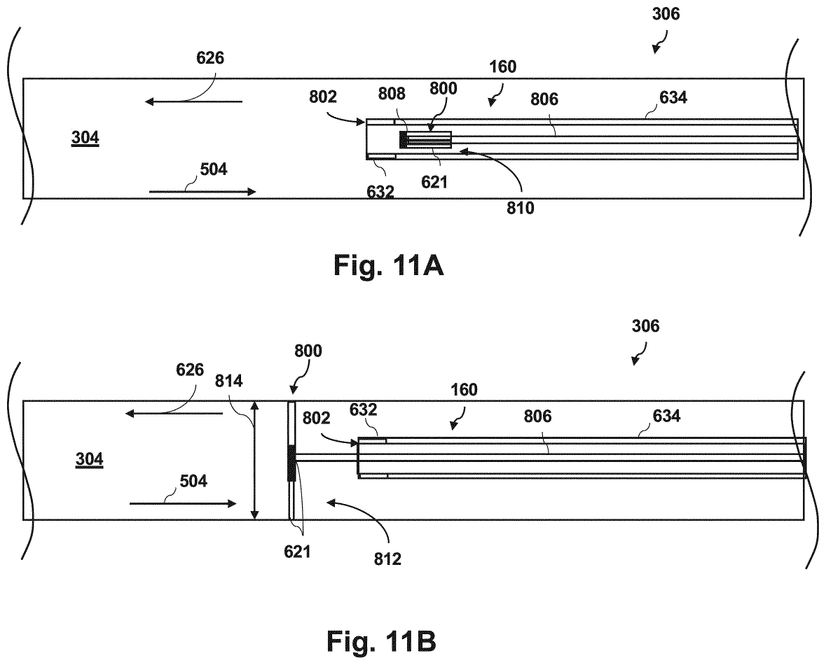

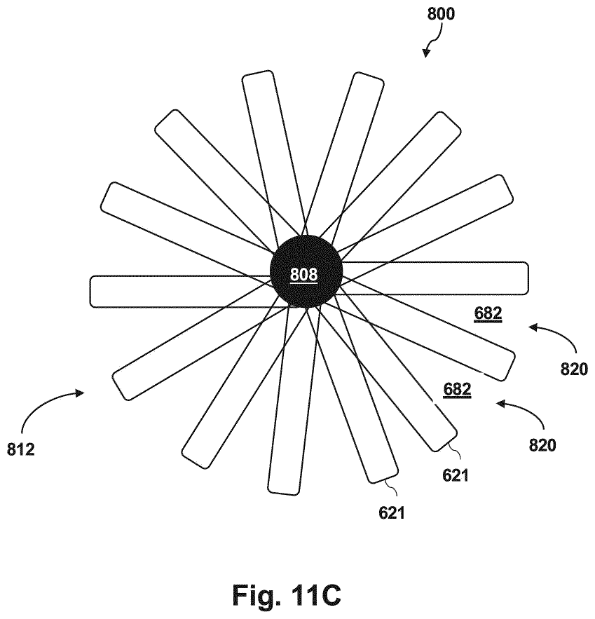

Turning now to FIGS. 11A-11C, in some embodiments, the intravascular reperfusion therapy device 160 may be configured to deliver reperfusion therapy via an expandable structure 800, which may include one or more arms 621 that may be deployed (e.g., expanded) distal of a distal end 802 of the catheter 634. In particular, FIG. 11A is a cross-sectional side view of the expandable structure 800 positioned within the catheter 634 of the intravascular reperfusion therapy device 160, FIG. 11B is a cross-sectional side view of the expandable structure 800 in an expanded state and positioned distal of the distal end 802 of the catheter 634, and FIG. 11C is a front view of the expandable structure 800 in the expanded state illustrated in FIG. 11B. In that regard, while the method 700 of FIG. 7 is described with respect to delivery of reperfusion therapy via the expandable basket 620, the method 700 may additionally or alternatively be employed with the expandable structure 800, as described below. Further, while the intravascular reperfusion therapy device 160 with the expandable structure is illustrated as being positioned within a coronary vein 304 in FIGS. 11A-B, the intravascular reperfusion therapy device 160 with the expandable structure 800 may additionally or alternatively be positioned within and deliver reperfusion therapy at a coronary artery 302.

With reference now to FIG. 11A, the expandable structure 800 may be coupled to a flexible elongate member 806, such as a guidewire (e.g., guidewire 636), which may extend through the lumen 690 of the catheter 634. In some embodiments, the flexible elongate member 806 may be coupled to an actuator at a proximal portion of the intravascular reperfusion therapy device 160, which may control movement and/or positioning of the flexible elongate member 806 and the expandable structure 800. For instance, the actuator may include one or more knobs or gears that may be manually controlled (e.g., by a physician) or automatedly controlled by the processing system 110.

FIG. 11A further illustrates the expandable structure 800 positioned within the catheter 634 and arranged in a first expansion state 810 (e.g., a collapsed state). In some embodiments, the arms 621 of the expandable structure 800 may be positioned proximate to one another within the first expansion state 810. In particular, the arms 621 may be held proximate to one another based on an electrical signal or a lack thereof in the case that the arms 621 are formed with an electroactive polymer. Additionally or alternatively, in the case that the arms 621 are responsive to application of thermal energy (e.g., different temperature ranges), the arms 621 may be maintained within the first expansion state 810 via control of the temperature at the arms 621. Further, in some cases, expansion of the arms 621 may be restrained (e.g., limited) by the diameter of the lumen 690 such that the arms 621 remain in the first expansion state 810 while positioned within the catheter 634.

FIG. 11B illustrates the expandable structure 800 in a second expansion state 812. As illustrated, in the second expansion state 812, at least a portion of the expandable structure 800 may be positioned distal of the distal end 802, and the arms 621 may expand within the blood vessel (e.g., a coronary vein 304). In particular, the arms 621 may expand to a diameter 814, which may substantially match a diameter of the blood vessel such that blood flow through the vessel is obstructed.

In some embodiments, the expandable structure 800 may manually (e.g., via user control) or automatedly (e.g., via control by the processing system 110) transition from the first expansion state 810 to the second expansion state 812 or vice versa. For instance, the processing system 110 may control a position (e.g., actuate) the flexible elongate member 806 so that the expandable structure 800 is positioned within or distal of the catheter 634. In some embodiments, such as cases where the lumen 690 limits the diameter of the arms 621, the arms 621 may be arranged to expand once the arms 621 are positioned distal of (e.g., spaced from) the catheter 634. To that end, the arms 621 may relax to the second expansion state 812 after leaving the confines of the catheter 634. Additionally or alternatively, the processing system 110 may control an electrical signal or a thermal energy applied to the arms 621 to transition the expandable structure 800 between expansion states. Moreover, the processing system 110 may be configured to oscillate the expandable structure 800 between the first expansion state 810 and the second expansion state 812 to control delivery of reperfusion therapy (e.g., at step 706 of the method 700) based on physiological data obtained from the sensor 632 (e.g., at step 702 of the method). Further, as described with respect to the expandable basket 620, the processing system 110 may additionally or alternatively control the degree of expansion of the expandable structure 800 to control delivery of the reperfusion therapy. In that regard, the processing system 110 may selectively actuate individual or subsets of arms 621 to control the degree of expansion of the expandable structure 800. Additionally or alternatively, the processing system 110 may control the expandable structure 800 to expand to different diameters to fully or partially obstruct blood flow. In any case, the degree of obstruction resulting from the various configurations of the expandable structure may correspond to different back pressures generated in the direction (indicated by arrow 626) opposite the direction (indicated by arrow 504) of blood flow and the resulting delivery of reperfusion therapy.

As illustrated in FIG. 11C, the arms 621 may be positioned at different circumferential locations about the flexible elongate member 806. Further, as illustrated in FIGS. 11A-C, the arms 621 may be coupled to a tip 808 of the expandable structure 800. In that regard, the arms 621 may be coupled to a common location, such as a common position with respect to the longitudinal axis of the intravascular reperfusion therapy device 160. In other embodiments, the arms 621 may be coupled to different positions along the flexible elongate member 806 with respect to the longitudinal axis of the intravascular reperfusion therapy device 160. That is, for example, the arms 621 may be longitudinally spaced from one another. For instance, a first arm 621 may be proximal of a second arm 621 along the flexible elongate member 806. Accordingly, in such embodiments, varying the position of the flexible elongate member 806 with respect to the catheter 634 may vary the degree of expansion (e.g., the degree of obstruction) provided by the expandable structure 800. For instance, in a first position, the second arm 621 may be distal of the distal end 802, while the first arm 621 may remain positioned in the catheter 634, resulting in a first level of obstruction to blood flow. In a different, second position, both the first and second arms 621 may be distal of the distal end 802, resulting in a greater, second level of obstruction to blood flow. Accordingly, the processing system 110 may control the position of the flexible elongate member 806 to control expansion of the arms 621 based on the progression of the reperfusion therapy (e.g., at step 706 of the method 700) in some embodiments.

As further illustrated by FIG. 11C, the expansion of the expandable structure 800 to the second expansion state 812 may result in the arms 621 being spaced from one another. In particular, gaps the arms 621 may be spaced from one another by the gaps 820. Further, in some embodiments, the gaps 820 may include empty space (e.g., may be unfilled), which may allow blood to flow through the gaps 820. Accordingly, in such embodiments, even when the expandable structure 800 is expanded to a diameter matching a diameter of the blood vessel, as shown in FIG. 11B, the expandable structure 800 may only partially obstruct blood flow through the blood vessel. In some embodiments, a material 682, such as a polymer, metal, composite material, and/or other biologically compatible material, may be coupled between the arms 621. More specifically, the material 682 may be coupled between the arms 621 such that expansion of the arms 621 (e.g., to the second expansion state 624) expands the material into the blood vessel. In particular, the material 682 may be arranged between the arms 621 such that the gaps 820 are partially or totally obstructed by the material. The material 682 may additionally or alternatively prevent blood flow through the lumen 690 of the catheter 634. Further, in cases where the material fully obstructs the gaps 680 when the expandable structure 800 is expanded to a diameter matching a diameter of the blood vessel, the expandable structure 800 may thus fully obstruct blood flow within the blood vessel.

FIGS. 12A-12B illustrate exemplary arrangements of the one or more sensors (e.g., first sensor 630 and/or second sensor 632) of the intravascular reperfusion therapy device 160. In particular, FIGS. 12A-12B illustrate a front view of a cross section of a catheter 634 of the intravascular reperfusion therapy device 160. In an exemplary embodiment, FIG. 12A illustrates a front view of the first sensor 630 positioned on a distal end of the catheter 634, where the catheter 634 terminates. In other embodiments, the first sensor 630 and the illustrated cross section of FIG. 12A may be spaced from the distal end of the catheter. Further, in an exemplary embodiment, FIG. 12B illustrates a front view of the first sensor 630 and the second sensor 632 positioned on a distal end of the catheter 634. In other embodiments, the first sensor 630, the second sensor 632, and the illustrated cross section may be spaced from the distal end of the catheter 634. Moreover, while FIGS. 12A-12B are illustrated and described with respect to positioning of sensors on a catheter, embodiments are not limited thereto. In that regard, the illustrated sensors may additionally or alternatively be arranged on a guidewire according to the techniques described herein.

In some embodiments, the one or more sensors of the intravascular reperfusion therapy device 160 may include one or more transducers, such as one or more ultrasound transducer elements. To that end, the illustrated configurations of FIGS. 12A-12B may be representative of one or more transducers. The one or more ultrasound transducer elements (e.g., acoustic elements) may be configured to emit ultrasound energy and receive echoes corresponding to the emitted ultrasound energy. Further, the one or more ultrasound transducer elements may include a piezoelectric/piezoresistive element, a piezoelectric micromachined ultrasound transducer (PMUT) element, a capacitive micromachined ultrasound transducer (CMUT) element, and/or any other suitable type of ultrasound transducer element. The one or more ultrasound transducer elements may further be in communication with (e.g., electrically coupled to) electronic circuitry. For example, the electronic circuitry can include one or more transducer control logic dies. The electronic circuitry can include one or more integrated circuits (IC), such as application specific integrated circuits (ASICs). In some embodiments, one or more of the ICs can include a microbeamformer (μBF). In other embodiments, one or more of the ICs includes a multiplexer circuit (MUX).

Further the one or more transducers of the one or more sensors may be arranged in any suitable configuration. For example, the transducers may be positioned in an array of ultrasound transducer elements, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of transducer elements (e.g., one or more rows, one or more columns, and/or one or more orientations) can be uniformly or independently controlled and activated. The array can be configured to obtain one-dimensional, two-dimensional, and/or three-dimensional images of patient anatomy.

In an exemplary embodiment, the first sensor 630 or the second sensor 632 is a flow sensor, which includes a single ultrasound transducer element, such as the transducer elements described above. The transducer element emits ultrasound signals and receives ultrasound echoes reflected from anatomy (e.g., flowing fluid, such as blood). The transducer element generates electrical signals representative of the echoes. These electrical signals are carried from the sensor at the distal portion of the device 160 to a connector at the proximal portion of the device 160, which may be communicatively coupled to the processing system 110. The processing system 110 can then process the electrical signals to extract the flow velocity of the fluid.

In some embodiments, the first sensor 630 or the second sensor 632 is thermal anemometric sensor, such as a thermal anemometric flow sensor. In such cases, the sensor may be maintained at a relatively constant temperature, and the electrical power consumed to maintain this temperature as blood flows proximate the sensor may be sensed and as representative of the flow velocity of the blood flow. Alternatively, a relatively constant power may be supplied to the sensor, and a change in temperature of the sensor as the blood flows proximate the sensor may be sensed as representative of the flow velocity of the blood flow. For instance, the processing system 110 may relate the consumed power or the temperature change, respectively to determine the flow velocity of the blood. While the sensors are described as being implemented with transducer elements or as a thermal anemometric sensor, embodiments are not limited thereto. In that regard, any suitable sensor configured to obtain physiological data associated with blood flow may be utilized in the intravascular reperfusion therapy device 160.

As shown in FIG. 12A, the first sensor 630 is arranged in an annular configuration about (e.g., following an outer perimeter of) the catheter 634. This annular configuration may include one or more transducers, such as one or more ultrasound transducers, as described above. Further, in some embodiments, the transducer elements and/or the sensor 630 may be forward-facing. That is, for example, the first sensor 630 may be a flow sensor arranged so that the first sensor 630 senses (e.g., emits signals) along the axis blood flows along within the vessel (e.g., the axis including the blood flow directions indicated by arrow 502 and arrow 504 in FIGS. 4-5 and 8-9), which may correspond to the longitudinal axis of the intravascular reperfusion therapy device). In that regard, the first sensor 630 may be arranged to sense in a direction aligned with or opposite the direction of blood flow within the vessel. In some embodiments, the sensor 630 may be substantially perpendicular with the longitudinal axis of the intravascular reperfusion therapy device 160 to be forward-facing. In some embodiments, the sensor 630 may be positioned at an oblique angle (e.g., tilted) such that at least a portion of the signals emitted by the sensor 630 detect physiological data with respect to the axis that blood flows along within the vessel. For instance, in cases where the first sensor 630 is spaced from the distal end of the intravascular reperfusion therapy device 160, the first sensor 630 may be positioned at an angle to detect blood flow.

FIG. 12B, illustrates the first sensor 630 and a second sensor 632 positioned on the catheter 634. Again, the illustrated first sensor 630 may represent one or more transducers, and the illustrated second sensor 632 may represent one or more transducers. Moreover, the first sensor 630 and the second sensor 632 may be the same or different. For instance, the first sensor 630 may be a flow sensor, as described above, and the second sensor 632 may be a flow sensor or a pressure sensor.

In some embodiments, the first sensor 630 and the second sensor 632 may be spaced from one another along the circumference of the catheter 634, as illustrated. Further, the first sensor 630 and the second sensor 632 may face the same or different directions. For instance, the first sensor 630 may be entirely forward facing (e.g., perpendicular to the longitudinal axis of the device 160), while the second sensor 632 may be tilted or parallel with the axis of the device 160. The second sensor 632 may be a pressure sensor arranged parallel with the axis of the device 160, for example.

Further, in some embodiments, the first sensor 630 and the second sensor 632 may be positioned within the catheter 634, such as within the lumen 690 of the catheter 634 and/or within a housing positioned within or defined by the lumen 690. In some embodiments, the first sensor 630 and the second sensor 632 may be embedded within a material of the catheter 634 or positioned on an outer surface of the catheter 634. In that regard, the first sensor 630 and the second sensor 632 may be arranged in any suitable configuration for collecting physiological data intravascularly.

While the system 100 and the method 700 are described herein as being employed for evaluating (e.g., assessing) and/or controlling reperfusion therapy, embodiments are not limited thereto. In that regard, the techniques described herein may additionally or alternatively be applied to microvascular disease (e.g., affecting capillary beds, for example) in any portion of a patient's anatomy (e.g., within or separate from the heart) and/or to nonobstructive coronary artery disease. Moreover, assessment and/or control of blood flow through particular tissue and/or capillary beds may be performed with or without a PCI therapy being performed on a vessel associated with the tissue and/or capillary beds.

A person of ordinary skill in the art will recognize that the present disclosure advantageously provides a system and method suitable to evaluate (e.g., assess) and control (e.g., adjust) a reperfusion therapy associated with an area of tissue. In particular, the techniques described herein enable adaptive delivery of a reperfusion therapy via an intravascular expandable structure with one or more arms based on intravascularly sensed physiological data. The logical operations making up the embodiments of the technology described herein are referred to variously as operations, steps, objects, elements, components, regions, etc. Furthermore, it should be understood that these may occur in any order, unless explicitly claimed otherwise or a specific order is inherently necessitated by the claim language.

It should further be understood that the described technology may be employed in a variety of different applications, including but not limited to human medicine, veterinary medicine, education and inspection. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal imaging system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
an intravascular reperfusion therapy device configured to be positioned within a coronary vein of a patient to deliver reperfusion therapy to a myocardium of a heart of the patient associated with the coronary vein, wherein the intravascular reperfusion therapy device comprises a catheter and a sensor, wherein the catheter comprises a plurality of arms configured to expand into the coronary vein to deliver the reperfusion therapy by obstructing blood flow in a first direction in the coronary vein to generate back pressure in an opposite, second direction within the coronary vein; and
a processor circuit in communication with the intravascular reperfusion therapy device and configured to:
receive, from the sensor, physiological data associated with blood flow through the coronary vein;
determine, based on the physiological data, a progression of the reperfusion therapy delivered to the myocardium of the heart; and
control, based on the progression of the reperfusion therapy, expansion of the plurality of arms while the intravascular reperfusion therapy device is positioned within the coronary vein such that the back pressure within the coronary vein is controlled.

2. The system of claim 1, wherein, to determine the progression of the reperfusion therapy, the processor circuit is configured to:
determine a derivative of the physiological data with respect to time.

3. The system of claim 1, wherein, to control the expansion of the plurality of arms, the processor circuit is configured to control a degree of expansion of the plurality of arms, wherein the degree of expansion corresponds to at least one of a quantity of expanded arms of the plurality of arms or a diameter to which the expanded arms are expanded.

4. The system of claim 3, wherein, to control the expansion of the plurality of arms, the processor circuit is configured to control an oscillation of the degree of expansion between a first degree of expansion and a different, second degree of expansion.

5. The system of claim 4, wherein the processor circuit is configured to control a frequency of the oscillation.

6. The system of claim 4, wherein the processor circuit is configured to control a duty cycle of the oscillation.

7. The system of claim 1, wherein, when expanded, the plurality of arms is configured to be spaced from one another such that a gap is positioned between a first and second arm of the plurality of arms.

8. The system of claim 1, wherein the intravascular reperfusion therapy device comprises a material coupled between the plurality of arms, wherein the expansion of the plurality of arms results in expansion of the material into the coronary vein.

9. The system of claim 1, wherein the intravascular reperfusion therapy device comprises:
a first expansion state in which the plurality of arms is unexpanded; and
a second expansion state in which at least one of the plurality of arms is expanded into the coronary vein.

10. The system of claim 9, wherein the plurality of arms comprise an electroactive polymer configured to deform responsive to an electrical signal, wherein the processor circuit is configured to control the electrical signal to transition the intravascular reperfusion therapy device from the first expansion state to the second expansion state.

11. The system of claim 9, wherein the plurality of arms comprise material configured to deform responsive to application of thermal energy, wherein the processor circuit is configured to control the thermal energy to transition the intravascular reperfusion therapy device from the first expansion state to the second expansion state.

12. The system of claim 9, wherein, in the first expansion state, the plurality of arms is positioned within a lumen of the catheter, and, in the second expansion state, the plurality of arms is positioned distal of a distal end of the catheter.

13. The system of claim 9, wherein the plurality of arms is integrally formed in the catheter such that the intravascular reperfusion therapy device is configured to buckle a portion of the catheter from a first position to a different, second position to transition from the first expansion state to the second expansion state.

14. The system of claim 1, wherein the processor circuit is further configured to:
output, to display in communication with the processor circuit, a visual representation of the progression of the reperfusion therapy.

15. The system of claim 1, wherein the sensor comprises a flow sensor, wherein the physiological data comprises a blood flow rate.

16. The system of claim 1, wherein the sensor comprises a pressure sensor, wherein the physiological data comprises pressure data.

17. The system of claim 1, wherein the sensor comprises an ultrasound transducer positioned at a distal portion of the catheter.

18. The system of claim 1, wherein the intravascular reperfusion therapy device further comprises a guidewire, wherein the sensor is positioned at a distal portion of the guidewire.

19. A system, comprising:
an intravascular reperfusion therapy device configured to be positioned within a coronary vein of a patient to deliver reperfusion therapy to a myocardium of a heart of the patient associated with the coronary vein, wherein the intravascular reperfusion therapy device comprises:
a flow sensor positioned at a distal portion of the intravascular reperfusion therapy device; and
a catheter comprising a plurality of arms, wherein the plurality of arms is configured to expand into the coronary vein to deliver the reperfusion therapy by obstructing blood flow in a first direction in the coronary vein to generate back pressure in an opposite, second direction within the coronary vein; and
a processor circuit in communication with the intravascular reperfusion therapy device and configured to:
receive, from the flow sensor, flow data associated with blood flow through the coronary vein;
determine, based on the flow data, a progression of the reperfusion therapy delivered to the myocardium of the heart; and
control, based on the progression of the reperfusion therapy, a degree of expansion of the plurality of arms while the intravascular reperfusion therapy device is positioned within the coronary vein such that the back pressure within the coronary vein is controlled, wherein the degree of expansion corresponds to at least one of a quantity of expanded arms of the plurality of arms or a diameter to which the expanded arms are expanded.

20. An apparatus, comprising:
an intravascular reperfusion therapy device configured to be positioned within a coronary vein of a patient to deliver reperfusion therapy to a myocardium of a heart of the patient associated with the coronary vein, wherein the intravascular reperfusion therapy device comprises:
a catheter;
a plurality of arms coupled to a distal portion of the catheter, wherein the plurality of arms is configured to expand into the coronary vein to deliver the reperfusion therapy by obstructing blood flow in a first direction in the coronary vein to generate back pressure in an opposite, second direction within the coronary vein; and
a control mechanism coupled to a proximal portion the catheter, wherein the control mechanism is configured to control a degree of expansion of the plurality of arms while the intravascular reperfusion therapy device is positioned within the coronary vein such that the back pressure within the coronary vein is controlled, wherein the degree of expansion corresponds to at least one of a quantity of expanded arms of the plurality of arms or a diameter to which the expanded arms are expanded.
